(19) United States
(12) Patent Application Publication
(10) Pub. No.: US 2016/0055304 A1
(43) Pub. Date: Feb. 25, 2016

(54) TARGETED ELECTRICAL STIMULATION

(71) Applicant: Aaken Laboratories, Davis, CA (US)

(72) Inventors: Michael J. Russell, Davis, CA (US); Qiang Wang, Davis, CA (US); David F. Wiley, Woodland, CA (US)

(21) Appl. No.: 14/930,485

(22) Filed: Nov. 2, 2015

Related U.S. Application Data
(63) Continuation-in-part of application No. 13/112,934, filed on May 20, 2011.

(51) Int. Cl. G06F 19/00; A61N 1/05; A61B 5/055

(52) U.S. Cl. CPC G06F 19/3412; G06F 19/3481; A61B 5/055; A61N 1/0529

(57) ABSTRACT

Apparatuses, systems, methods, and program products are disclosed for targeted electrical stimulation. A method includes determining, by a processor, a base configuration for each of a plurality of electrodes configured to transmit an electrical current towards a target area of a biological structure. A method includes calculating a cost associated with transmitting an electrical current to the target area according to the base configuration. A method includes modifying the base configuration in response to the calculated cost not satisfying a minimum cost threshold. The modified configuration clusters electrical current transmitted by each of the electrodes at the target area according to a resistivity map of the biological structure to minimize the cost of transmitting the electrical current. The cost is re-calculated according to the modified configuration and the configuration is modified until the minimum cost threshold is satisfied. A method includes graphically presenting the configuration for each of the electrodes.

### CROSS-REFERENCES TO RELATED APPLICATIONS

This is a continuation-in-part application of and claims priority to U.S. patent application Ser. No. 13/112,934 entitled “Methods and Systems for Generating Electrical Property Maps of Biological Structures” and filed on May 20, 2011, for Michael J. Russell, which claims priority to U.S. patent application Ser. No. 11/424,813 filed on Jun. 16, 2006, which claims priority to U.S. Provisional Patent Application No. 60/691,068 filed on Jun. 16, 2005, all of which are incorporated herein by reference.

### FIELD

This invention relates to electrical stimulation and more particularly relates to targeted electrical stimulation using electrical clustering by current summation.

### BACKGROUND

Electrical stimulation may be used to treat certain conditions, such as Parkinson's disease, epilepsy, depression, or the like. Electrodes may be placed at predefined locations on a biological structure, such as a head, and electrical current may be transmitted from the electrodes into the biological structure and towards an area of interest, such as an area of the brain. However, it may be the case that the electrical current used to stimulate an area of interest of the biological structure may also affect other areas of the biological structure, which may cause unintended side effects.

### SUMMARY

An apparatus for targeted electrical stimulation is disclosed. A system and method also perform the functions of the apparatus. An apparatus, in one embodiment, includes a basis module that determines a base configuration for each of a plurality of electrodes. In certain embodiments, the electrodes are configured to transmit an electrical current towards a target area of a biological structure.

An apparatus, in some embodiments, includes a cost module that calculates a cost associated with transmitting an electrical current to the target area according to the base configuration. In a further embodiment, an apparatus includes an adjustment module that modifies the base configuration for each of the electrodes in response to the calculated cost not satisfying a minimum cost threshold. In one embodiment, the modified configuration clusters electrical current transmitted by each of the electrodes at the target area according to a resistivity map of the biological structure such that the cost of transmitting the electrical current is minimized.

In a further embodiment, the cost module re-calculates the cost according to the modified configuration and the adjustment module modifies the configuration of each of the electrodes until the minimum cost threshold is satisfied. In certain embodiment, an apparatus includes a display module that graphically presents the configuration for each of the electrodes that minimizes the calculated cost of transmitting the electrical current towards the target area.

A method, in one embodiment, includes determining, by a processor, a base configuration for each of a plurality of electrodes. In some embodiments, the electrodes are configured to transmit an electrical current towards a target area of a biological structure. In a further embodiment, the method includes calculating a cost associated with transmitting an electrical current to the target area according to the base configuration.

In some embodiments, the method includes modifying the base configuration for each of the electrodes in response to the calculated cost not satisfying a minimum cost threshold. In one embodiment, the modified configuration clusters electrical current transmitted by each of the electrodes at the target area according to a resistivity map of the biological structure such that the cost of transmitting the electrical current is minimized. In certain embodiments, the cost is re-calculated according to the modified configuration and the configuration of each of the electrodes is modified until the minimum cost threshold is satisfied. In some embodiments, the method includes graphically presenting the configuration for each of the electrodes that minimizes the calculated cost of transmitting the electrical current towards the target area.

A computer program product, in one embodiment, includes a computer readable storage medium having program code embodied therein. In one embodiment, the program code is readable/executable by a processor for determining a base configuration for each of a plurality of electrodes. In one embodiment, the electrodes are configured to transmit an electrical current towards a target area of a biological structure.

In a further embodiment, the program code is readable/executable by a processor for calculating a cost associated with transmitting an electrical current to the target area according to the base configuration. In some embodiments, the program code is readable/executable by a processor for modifying the base configuration for each of the electrodes in response to the calculated cost not satisfying a minimum cost threshold.

In one embodiment, the modified configuration clusters electrical current transmitted by each of the electrodes at the target area according to a resistivity map of the biological structure such that the cost of transmitting the electrical current is minimized. In a further embodiment, the cost is re-calculated according to the modified configuration and the configuration of each of the electrodes is modified until the minimum cost threshold is satisfied.

In some embodiments, the program code is readable/executable by a processor for graphically presenting the configuration for each of the electrodes that minimizes the calculated cost of transmitting the electrical current towards the target area.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1A illustrates one embodiment of a transcranial motor evoked potential (tcMEP);

FIG. 1B illustrates another embodiment of a tcMEP;

FIG. 2 illustrates an embodiment of a human head with materials of different conductivities conventionally identified and having two electrodes coupled therewith;

FIG. 3 illustrates an embodiment of a human brain having a mesh for finite element modeling applied thereto;

FIG. 4 illustrates an embodiment of a human brain having several tissue compartments identified and segmented;

FIG. 5 illustrates an embodiment of a human brain having several tissue compartments having different anisotropic resistivities identified and segmented, and having a mesh for anisotropic finite element modeling applied thereto;

FIG. 6a illustrates an embodiment of a human brain with two selected electrode locations and a current path defined therein;

FIG. 6b illustrates an embodiment of the human brain of FIG. 6a having a mesh for finite element modeling applied thereto;

FIG. 6c illustrates an embodiment of the human brain of FIG. 6b with anisotropies ascribed to elements of the mesh;

FIG. 6d illustrates examples of plots of current density through identical regions of isotropic and anisotropic models;

FIG. 7a illustrates examples of current density variations around areas of varying isotropic resistivities;

FIG. 7b illustrates a finite element mesh with mesh elements of different sizes and shapes;

FIG. 8 illustrates examples of various T1, T2, and PD MRI images;

FIG. 9 illustrates an example of a combined MRI image and a histogram of pixel intensities in the combined MRI image;

FIG. 10 illustrates an embodiment of three-dimensional modeling of current densities applied to a human brain coupled with two electrodes;

FIG. 11 illustrates an embodiment of a method for generating a combined image in accordance with some embodiments;

FIG. 12 is a block diagram illustrating an embodiment of a system for targeted electrical stimulation;

FIGS. 13A and 13B depict a schematic flow chart diagram illustrating an embodiment of a method for generating an electrical property map;

FIG. 14 depicts a schematic flow chart diagram illustrating an embodiment of a method for providing electrical or magnetic stimulation of a biological structure;

FIG. 15 depicts a schematic flow chart diagram illustrating an embodiment of a method for killing cells;

FIG. 16 depicts a schematic flow chart diagram illustrating an embodiment of a method for classifying tissues;

FIG. 17 illustrates an embodiment of a module for targeted electrical stimulation;

FIG. 18 illustrates an example of a predefined electrode placement model;

FIG. 19 illustrates a comparison of targeted and standard electrical stimulation; and

FIG. 20A illustrates an interface for targeted electrical stimulation;

FIG. 20B illustrates an embodiment of an MRI depicted tissue within a brain;

FIG. 20C illustrates an embodiment of stimulating tissue within a brain using electrical clustering; and

FIG. 21 illustrates a schematic flow chart diagram for one embodiment of a method for targeted electrical stimulation.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims, or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

### ABBREVIATIONS

CT=X-ray Computed Tomography

DWI=Diffusion Weighted MRI

DTI=Diffusion Tensor Tracking

MRA=Magnetic Resonance Angiography

GETs=Guided Electrical Transcranial stimulation

EEG=Electroencephalogram

MRI=Magnetic Resonance Imaging

T1 MRI=T1-weighted MRI

T2 MRI=T2-weighted MRI

PD MRI=Proton density weighted MRI (also known as spin density weighted MRI)

FE=Finite Element method

SEP=Somatosensory Evoked Potentials

fMRI=functional Magnetic Resonance Imaging

tcMEP=transcranial Motor Evoked Potentials

### INTRODUCTION

Transcranial electrical stimulation to elicit motor evoked potentials (tcMEPs) has become the standard of care for monitoring the motor pathways of the spinal cord and brain during high risk surgeries. A conventional tcMEP technique can often be a crude, but effective, tool to monitor motor pathways and to identify iatrogenic injuries.

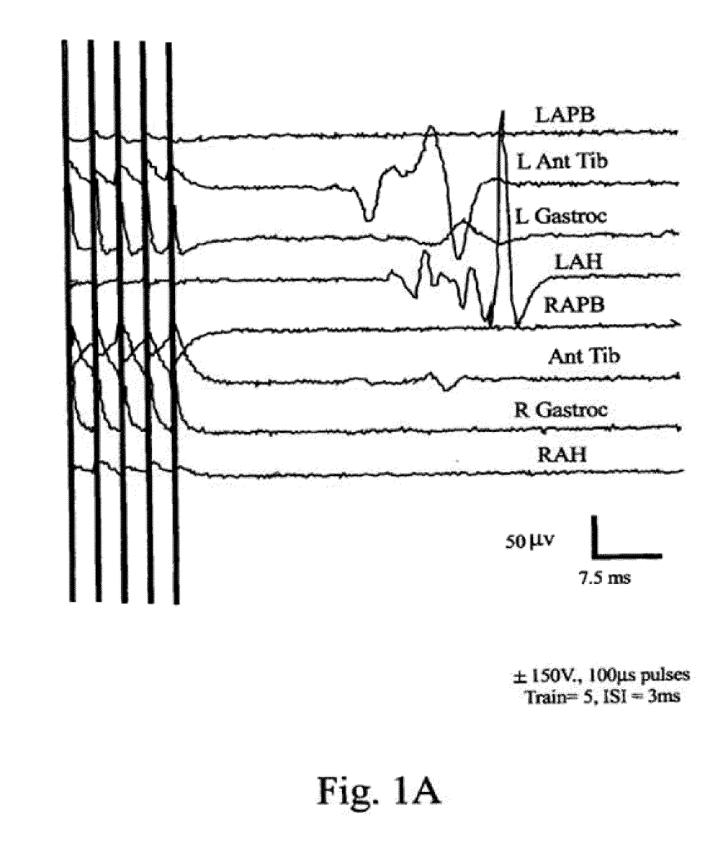

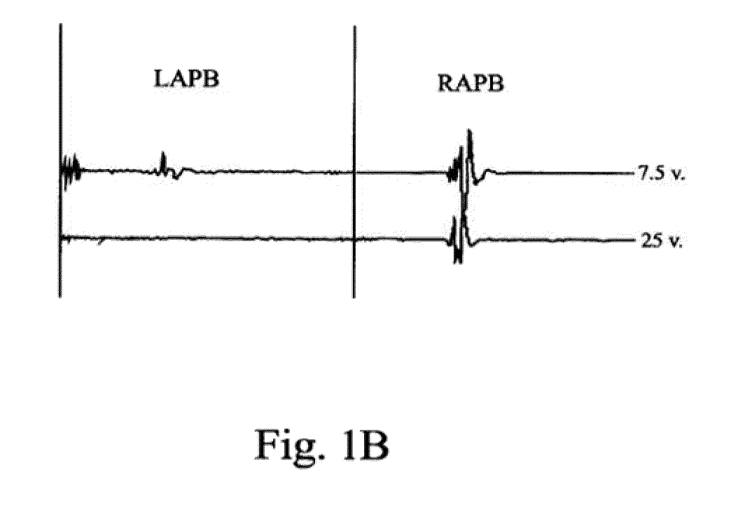

FIG. lA illustrates a tcMEP from a scoliosis patient. The scale of FIG. 1A shows 50 μV on the y-axis and 7.5 ms on the x-axis. Applied pulses were 150 Volts for 100 μs in trains of five pulses with an inter stimulus interval (ISI) of 3 ms. FIG. 1B illustrates a tcMEP from an 86 year old male with a neck fracture. Applied pulses were 75 Volts in the upper plot and 25 Volts in the lower plot.

Typically, a tcMEP procedure involves placing electrodes in the patient's scalp at locations that are thought to encompass the motor cortex and then applying brief high voltage electrical pulses with the intention of activating distal muscles or muscle groups.

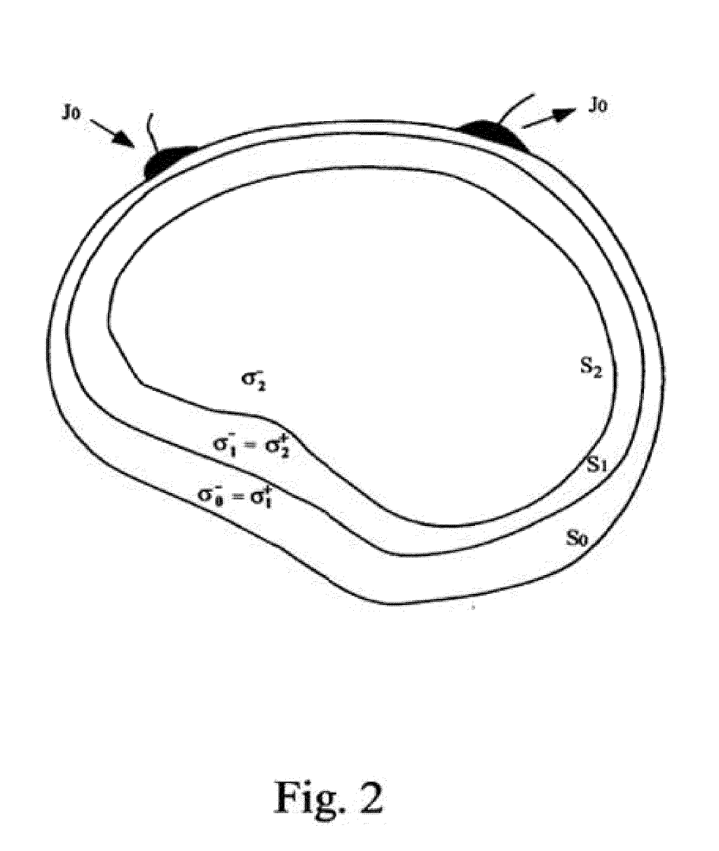

FIG. 2 illustrates placement of electrodes J0 outside of a patient's scalp. FIG. 2 also illustrates three regions S0, S1, and S2 having different conductivities σ1, σ2, and σ3, respectively. Unfortunately, the high voltages typically used to induce tcMEPs and the responses they produce can activate whole regions of the head, body, or trunk as well as the target muscles. The movement of large muscle groups due to the uncontrolled current spread means that seizures, broken jaws and patient movement create risk factors that have been associated with tcMEP testing. Applying stimulus trains rather than single pulses and adjustments in anesthesia techniques have significantly reduced the applied electrical currents used from 700-900 V to 200-400 V.

tcMEPs have become widely accepted as a less onerous substitute for “wake-up tests” in which the patient is awakened during surgery and asked to move their limbs before the surgical procedure is completed. However, these reduced stimulus levels may still exceed normal physiological levels and the uncontrolled movement of large muscle groups suggests that the applied pulses continue to result in significant current spreads. While major side effects may be relatively rare, tongue lacerations, muscle tears, and bucking are still rather common side effects. The large muscle movements that are sometimes associated with tcMEPs also limit the usefulness of the tcMEPs during periods when the surgeon is involved in delicate brain or spinal procedures.

It is desired to reduce or eliminate these side effects by predicting the paths of electrical pulses within the brain and adjusting current levels based on the predicted paths. It is also desired to reduce the current strength to near physiological levels at targeted areas to allow brain electrical stimulation to be used for treatment of patients outside of surgery. In this way, a significant positive impact may be achieved in the treatment of a number of disease conditions that have been demonstrated to benefit from brain electrical stimulation, e.g., Parkinson's disease, chronic pain, depression, and/or the like.

### Modeling

The head is a heterogeneous, anisotropic conductive medium with multiple conductive compartments. Finding the current path through this medium has been a significant problem in neurophysiology. For decades it has been the dream of many investigators to stimulate the brain through this medium without the use of brain surgery or depth electrodes. It is desired to provide an innovative solution to this problem.

Several attempts have been made to construct physical models of the head. Some of these physical models were made of plastic, saline and/or silicon, for example. They are not sufficient to represent the complexity of the problem and do not account for individuals' anatomical differences.

Finite element (FE) forward modeling has benefited from recent improvements in estimates of skull and tissue resistivity. These newer estimates were obtained in vivo and provide more precise values of indigenous tissues than many of the previous estimates that were typically done on dried or cadaver tissues.

Several groups have attempted to resolve the problem of transcranial stimulation by using commercially available transcranial magnetic stimulators. Although magnetic stimulators may be commonly used in clinics, they have been rejected for some applications because of the difficulty in using them in an environment with multiple metal objects and their tendency for the stimulation parameters to be less consistent than those produced by electrical stimulation. Small movements of the magnetic pulse generators have resulted in significant changes in the stimulus parameters and the coil cannot be used for chronic conditions wherein treatment would involve continuous stimulation. It is desired to accurately model head tissues and current pathways to more efficiently target cerebral activation of cortico spinal tract neurons by transcranial electrical stimulation.

As will be described in more detail below, solutions to the forward problem are achievable with matrix algebra by constructing a model of sufficient detail representing all the heterogeneities found within an individual's head and brain. The approach described below has bypassed the use of a physical model and uses an individual's MRI and/or X-ray computer tomography (CT) scan as a representation of the head and brain. MRI images and CT scans are digitized images that can be manipulated through computer programs to which standard algebraic manipulations can be applied. This digital modeling also allows the use of matrix algebra solutions that have been developed for other complex representations e.g. weather systems, fluid streams, etc. Further, modules within finite element (FE) analysis packages have been developed to represent time dependent factors such as capacitance and resistance.

It is further described below to advantageously reduce current densities by utilizing a three-dimensional (3-D) modeling of the head. In some embodiments, a two-dimensional (2-D) Guided Electrical Transcranial stimulation (GETs) is able to reduce current densities by 60 percent or more. Greater reduction may be achieved with the 3-D model.

Effective embodiments are provided, including combining CT scans with MRI images, for example. Such combinations can be advantageously utilized as a base for a GETs model. Computed Tomography (CT) is a particularly effective method of imaging various biological structures and is utilized in some embodiments for further enhancing the GETs model.

In one embodiment, direct measurements are obtained of current within subject brains. In another embodiment, motor evoked potentials are obtained as a biological assay. A technique in accordance with exemplary embodiments works advantageously in reducing electrical current densities even when brain anatomy has been significantly altered by an injury, tumor, or developmental disorder. In addition, GETs modeling can be applied to actual spinal surgery patients. This can serve to optimize transcranial stimulation of the motor cortex.

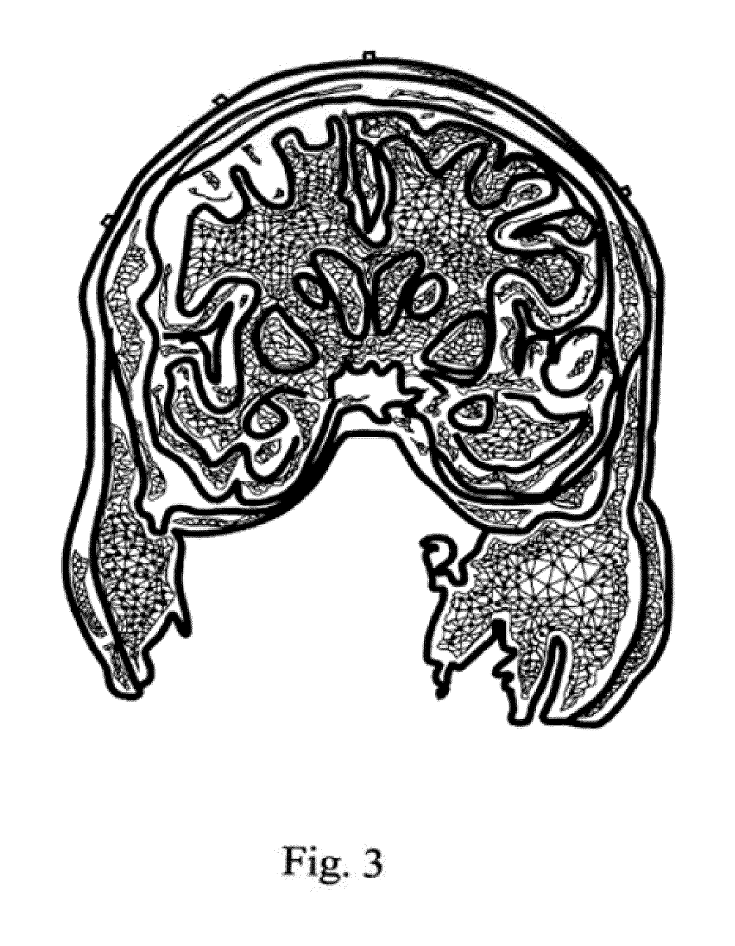

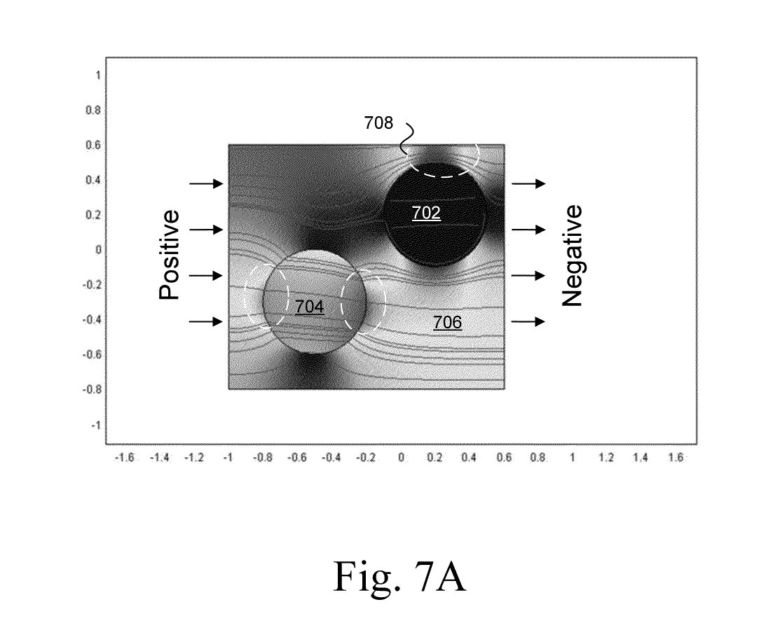

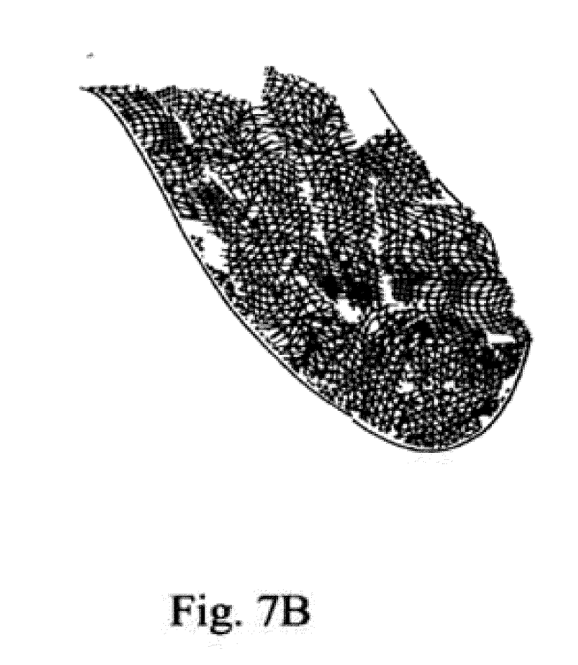

In some embodiments, a 2-D model has been developed of a single MRI slice through a head. FIG. 3 illustrates a human brain having a mesh for finite element modeling applied thereto (see also FIG. 7B which illustrates a finite element mesh with mesh elements of different sizes and shapes). The mesh includes elements of different shapes and sizes that have different resistivities assigned to them. In the 2-D embodiment, for example, current paths after transcranial stimulation can be predicted, e.g., in an anatomically correct coronal section through the upper limb representation of motor cortex, using FEM methods.

Current densities may be obtained in this embodiment for a coronal MRI section (6.5 mm) through the upper limb motor cortex. In one embodiment, the modeling proceeds in two steps: first, segmentation is performed to identify tissue compartment boundaries and resistivities, and, second, a finite element model is implemented to solve the forward problem (modeling measurements using given parameter values) for current densities.

### Segmentation

The scanned image may be contrast-enhanced and then preliminary tissue compartment boundaries may be identified automatically, semi-automatically, or manually, and in some cases, using commercially available software (e.g., Canvas).

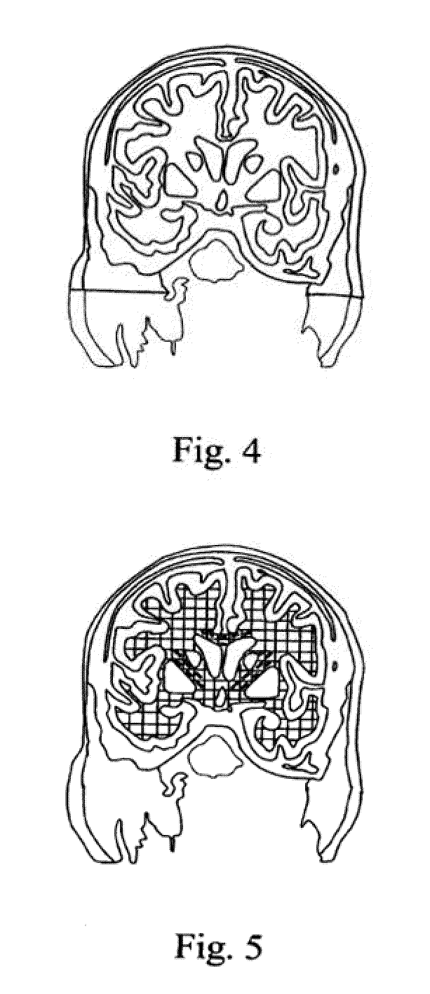

FIG. 4 illustrates a human brain having several tissue compartments identified and segmented according to their different resistivities in accordance with some embodiments. The tissue compartments that are segmented in the representation of FIG. 4 include cerebrospinal fluid (CSF) at 65 ohm-cm, white matter at 85 ohm-cm, blood at 160 ohm-cm, skin at 230 ohm-cm, gray matter at 300 ohm-cm, soft tissue at 500 ohm-cm, cancellous bone at 2500 ohm-cm, and compact bone at 16000 ohm-cm. The preliminary boundaries may be superimposed over an original MRI image, such as the MRI image illustrated in FIG. 5.

In the embodiment of FIG. 5, a grid is shown which serves as a finite element mesh, and the elements have directionalities or anisotropies ascribed thereto and illustrated with the slanted lines inside the elements of the grid. These directionalities correspond to directionalities of the nerve fibers.

### Finite Element Modeling

2-D current densities may be expressed as amps per meter, while 3-D current densities may be expressed in amps per square centimeter that would be applied in a 3-D model. Units of coulombs per square centimeter may also be used for modeling pulses.

Bilateral electrode placements (and an applied potential difference of 100 V) may be calculated for the segmented section, using an FE model generated using FEMLAB, for example. A mesh may be constructed by first detecting edge contours of each segment within the image, then converting the region within each contour into 2-D subdomains. Meshing of the entire structure may be carried out using standard FEMLAB meshing routines, requiring that minimum element quality be 0.1, (in one embodiment, a quality parameter may vary between 0 and 1 with an acceptable minimum mesh quality being 0.6). For example, the modal value of mesh quality may be approximately 0.98. Triangle quality is given by the formula: q=4°\sqrt{3a}÷(h₁²+h₂²+h₃²), where a is the triangle area and h₁, h₂, and h₃ are side lengths of the triangle; and q is a number between 0 and 1. If q>0.6, the triangle is of acceptable quality, and q=1 when h₁=h₂=h₃. If triangle elements have low q they are typically long and thin, which may result in the solution on the mesh being inaccurate. For example, the linear meshes for the model illustrated in FIG. 3 contained approximately 180,000 elements and 364,000 degrees of freedom.

### Modeling Results

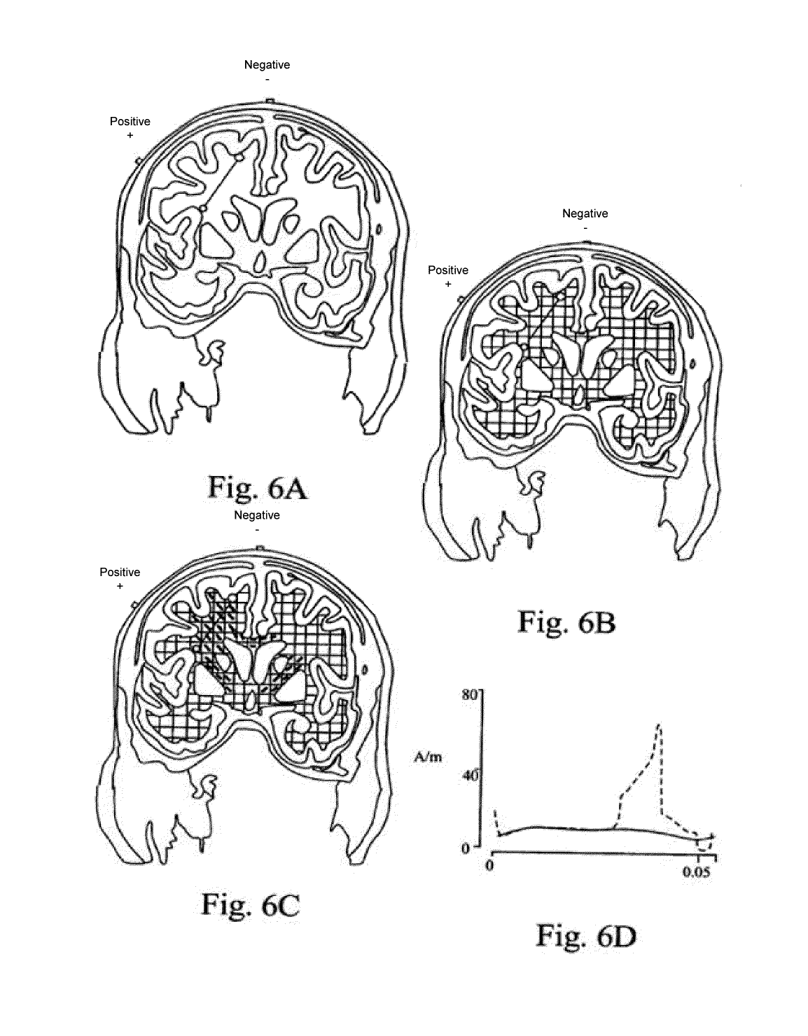

The modeling results are illustrated at FIGS. 6A-6D. FIG. 6A depicts a representation of a human brain with multiple compartments segmented by values of resistivity and having line boundaries obtained from an isotropic model. FIG. 6A also illustrates a pair of electrode locations “+” and “−.” A current path of interest (CPI) is also indicated in FIG. 6A.

The image of FIG. 6B has a matrix or grid of squares, rectangles, or other polygons such as triangles over the features illustrated in FIG. 6A. FIG. 6C illustrates the anisotropies as directional lines within at least some of the polygons that make up the grid.

The line plots in FIG. 6D are of current densities through identical locations along the CPI illustrated at FIGS. 6A, 6B, and 6C. The solid line in FIG. 6D is the current density obtained from the isotropic model represented at FIG. 6A, while the dashed line in FIG. 6D is the current density obtained from the anisotropic model of FIG. 6C. In the illustrated example, a peak P around 68 A/m was observed for the anisotropic model, while the isotropic model provided a maximum of 16 A/m for the homogeneous white matter region studied along the CPI. As can be seen from this result, tissue anisotropies have a significant influence on the location of the hot spots.

The GETs model, in certain embodiments, demonstrates some expected and unexpected results. As expected, there is a concentration of current below the electrodes. However, unexpectedly, the optimal current path demonstrated is not always the path of least resistance. There may be regions of high current density where there is a high conductivity inclusion within a sphere of lower conductivity (e.g., see zones at the pituitary stalk and the ventricle).

FIG. 7A illustrates this effect. In one embodiment, FIG. 7A illustrates a high-resistance area 702, a low-resistance area 704, and a medium resistance area 706. The effect appears, in one embodiment, to create “hot spots” 708 of electric field induced in the surrounding low conductivity region. In some embodiments, the current increase is greatest in the vicinity of interfaces that lie perpendicular to the current flow. Some of these current densities may be substantially above the surrounding area and significantly distant to the placement of the electrodes. In this context, the challenge may be to determine electrode locations such that unwanted activation is minimized, while stimulating targeted areas efficiently.

Tissue isotropy may be advantageously modeled in accordance with some embodiments, and it has been modeled for an injection current in the brain. Models of further embodiments include anisotropic modeling of blood vessels and directionality of muscle fibers. Because the GETs model is based on MRI images and/or CAT scans of individuals, it also adjusts to developmental and individual differences in brain structure. In one embodiment, the most significant of these are the differences in bone structure.

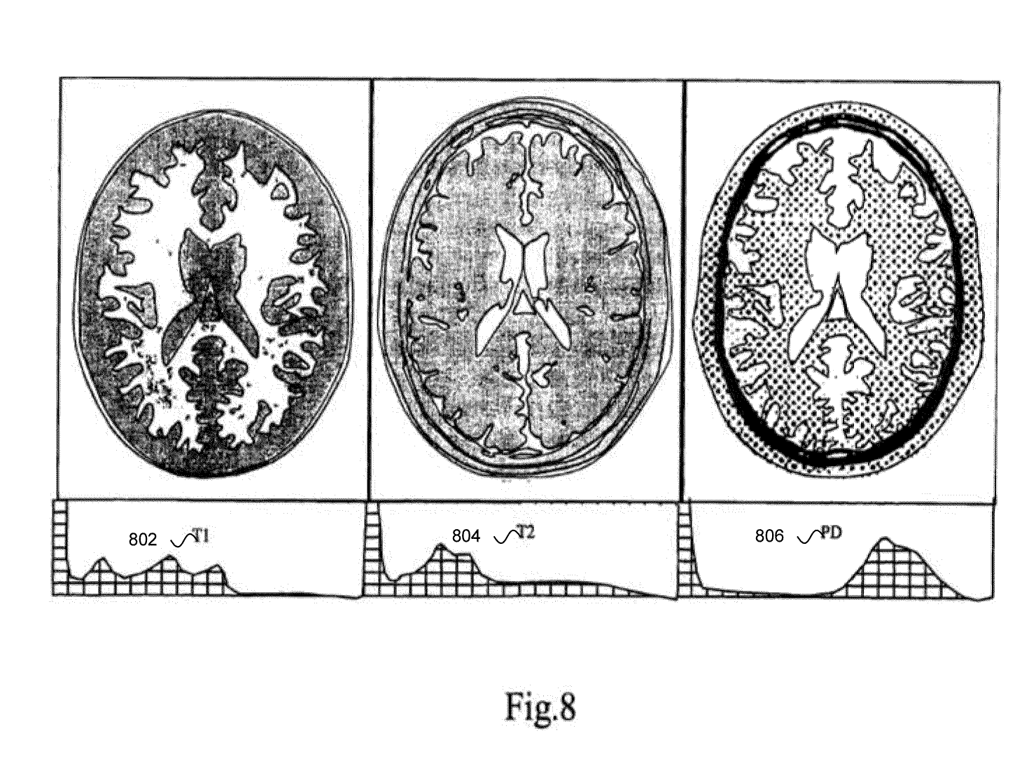

FIG. 8 illustrates MRI images of three different types: T1 802, T2 804, and PD 806. Below each MRI image 802-806 is a histogram of pixel intensities shown in the corresponding MRI image 802-806. As is well known to persons having ordinary skill in the art, each MRI image 802-806 includes multiple pixels (or voxels), and each pixel (or voxel) has a value (often called intensity). For images taken with an 8-bit resolution MRI instrument, each pixel value (or pixel intensity) may range from 0 to 255. The histogram for the T1 MRI image 802, for example, shows three peaks that may correspond to differences in resistivities of tissues within the brain. The histogram for the T2 MRI 804 image shows one, or possibly two, peaks, and the histogram for the PD MRI image 806 shows one peak at a different resistivity than T2 804 or T1 802. By utilizing information from multiple images 802-806 of different MRI types, it is possible to enhance segmentation based on pixel intensities. In certain embodiments, the histograms of the individual MRI images 802-806 do not distinguish between different types of tissue within the biological structure. In a further embodiment, as described below, a histogram of a combined image using the various MRI images 802-806, however, can be used to separate and/or distinguish different types of tissues and the corresponding resistivity values.

In some embodiments, utilizing information from multiple images (e.g., images of different MRI types) includes generating a combined image from the multiple images. One way to generate a combined image is by interleaving the multiple images.

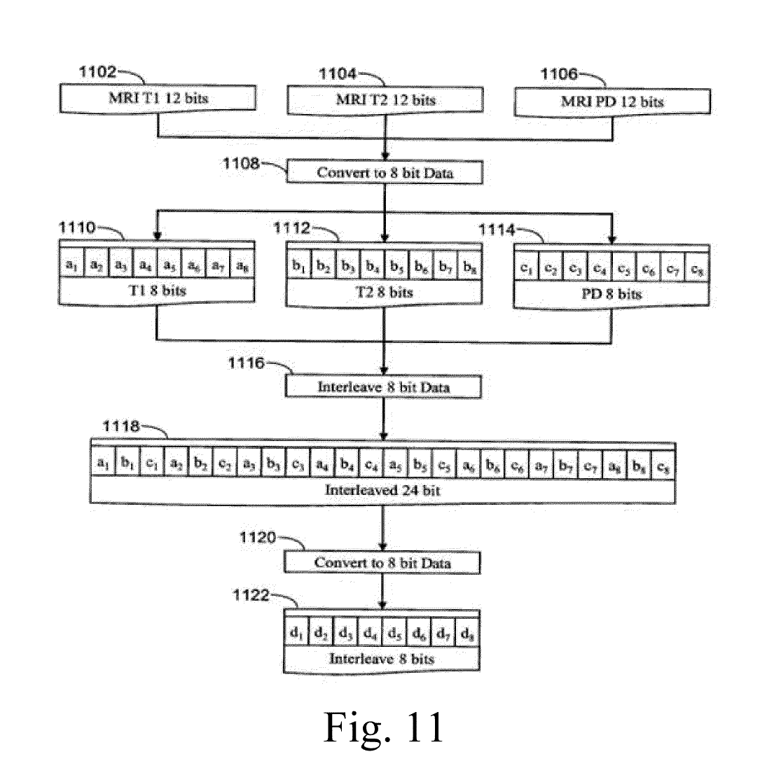

FIG. 11 illustrates one embodiment of a method of generating a combined image by interleaving multiple images in accordance with some embodiments. The method starts with multiple images (e.g., 1102, 1104, and 1106) as input. The multiple images may comprise any combination of two or more of: T1, T2, and proton density MRI images; a magnetic resonance angiography image; an X-ray computed-tomography image; or the like. Typically, three or more of T1, T2, and proton density MRI images; a magnetic resonance angiography image; and an X-ray computed-tomography image are used. For example, the multiple images include a T1 MRI image 1102, a T2 MRI image 1104, and a PD MRI image 1106.

Typically, the multiple images are registered (e.g., a same portion of each of the multiple images corresponds to a same portion of the biological structure). In some cases, raw images of a subject taken with a same MRI instrument in different image modes are registered. However, when the raw images are not registered (e.g., due to patient movement or multiple images taken with different instruments), one or more images of the raw images may be translated, rotated, and scaled to obtain the registered multiple images. The methods for registering the raw images are well known in the art, and thus are not repeated herein for brevity.

In addition, the multiple images may be normalized. In some embodiments, the normalization is performed by adjusting the intensity of each image so that the highest pixel value in each image corresponds to a predefined value. In certain embodiments, the normalization is performed by adjusting the intensity of each image so that a pixel corresponding to a certain biological medium (e.g., cerebrospinal fluid) has an intensity matching a predefined value.

In some embodiments, each MRI image is converted (1108) to an image of a predefined intensity resolution. For example, when each pixel of the multiple images (1102, 1104, and 1106) includes 12-bit data, such pixel can be converted into 8-bit data pixel. In some embodiments, the conversation is performed by selecting a predefined number of most significant bits (e.g., eight left-most bits for a conversion from 12-bit data to 8-bit data). In various embodiments, the conversion is performed based on normalization. For example, the 12-bit data is converted into the 8-bit data by dividing the 12-bit data with the maximum possible value of 12-bit data (e.g., 4095) and multiplying with the maximum possible value of 8-bit data (e.g., 255). As a result, converted images (e.g., 1110, 1112, and 1141) are obtained. The converted image 1110, which includes 8-bit data and corresponds to the T1 MRI image 1102, includes eight bits a₁ through a₈ for each pixel. The converted image 1112, which includes 8-bit data and corresponds to the T2 MRI image 1104, includes eight bits b₁ through b₈ for each pixel. The converted image 1114, which includes 8-bit data and corresponds to the PD MRI image 1106, includes eight bits c₁ through c₈ for each pixel.

In one embodiment, the converted images (e.g., 8-bit data), or the original images (e.g., 12-bit data) are interleaved (1116) to generate an interleaved image 1118. When interleaving three 8-bit converted images (e.g., 1110, 1112, and 1114), the interleaved image 1118 includes 24-bit data (e.g., [a₁, b₁, c₁, a₂, b₂, c₂, a₃, b₃, c₃, a₄, b₄, c₄, a₅, b₅, c₅, a₆, b₆, c₆, a₇, b₇, c₇, a₈, b₈, and c₈]).

In some embodiments, the interleaved image 1118 is converted into an image 1122. In some embodiments, the conversion is performed by selecting a predefined number of most significant bits (e.g., eight left-most bits for a conversion from 24-bit data to 8-bit data, such as [a₁, b₁, c₁, a₂, b₂, c₂, a₃, b₃, c₃]). In some other embodiments, the conversion is performed based on normalization. For example, the 24-bit data is converted into the 8-bit data by dividing the 24-bit data with the maximum possible value of 24-bit data (e.g., 16,777,215) and multiplying with the maximum possible value of 8-bit data (e.g., 255).

The output of the interleaving process may vary depending on the order of the multiple images. For example, a different order of the T1, T2, and PD MRI images may be used (e.g., T1, PD, and T2; T2, T1, and PD; T2, PD, and T1; PD, T1, and T2; PD, T2, and T1).

Alternatively, a weighted-sum image may be used. Each pixel (or voxel) in the weighted-sum image has an intensity based on a weighted-sum of intensities of corresponding pixels (or voxels) in the multiple images. In some embodiments, this relationship can be expressed as:

I_{c}=Σwⱼ*Iⱼ

where I_{c} is an intensity of a respective pixel in the combined image, wⱼ is a weight for a j-th image of the multiple images, and Iⱼ is an intensity of a corresponding pixel in the j-th image of the multiple images. In some embodiments, the intensity of the respective pixel in the combined image also includes higher-order terms (e.g., a second power and/or a third power of the intensity of the corresponding pixel).

In some embodiments, the weights (e.g., wⱼ) are determined using least-squares methods on a training set of images. The training set of images includes multiple images that include regions of known electrical properties.

In such embodiments, the intensity of each pixel in the combined image may directly correspond to an electrical property of a region of a biological structure corresponding to the pixel. For example, the intensity of each pixel in the weighted-sum image may represent an electrical property (e.g., resistivity) of the corresponding region (e.g., white matter) of the biological structure.

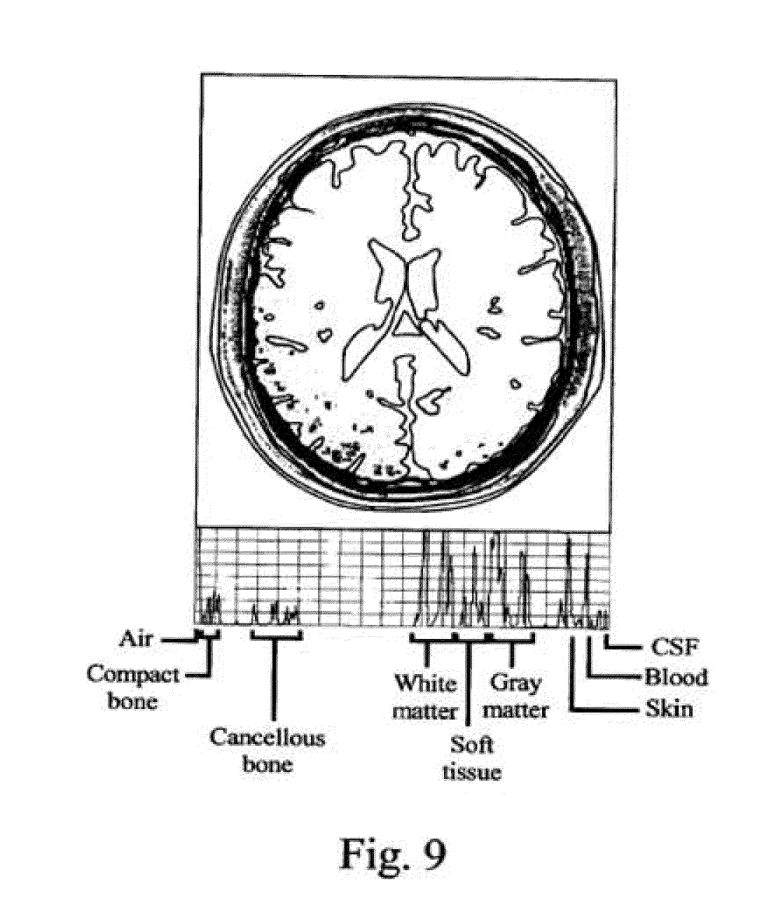

The combined image shown in FIG. 9 is an interleaved image of T1, T2, and PD MRI images of a brain. FIG. 9 is a prophetic example of a combined image and a histogram of pixel intensities (or voxel intensities) showing multiple peaks. The histogram may be generated based on pixels in the illustrated slice of an image.

Alternatively, the histogram may be generated based on selected voxels (e.g., all the voxels corresponding to a selected organ or region of a subject). The histogram for the combined image shown in FIG. 9 resolves multiple peaks corresponding to various tissue types including compact bone, cancellous bone, white matter, soft tissue, gray matter, skin, blood and cerebrospinal fluid. Other resolvable tissues may include cancerous tissue, inflammatory tissue and ischemic tissue, as well as eye fluid. By having enhanced resolution of tissues, it is possible to assign more correctly the resistivities or other electrical values of brain or other body tissues, and thereby calculate more precisely the current or other electrical input to be applied for monitoring or therapeutic purposes (e.g., for killing cells, such as tumor cells).

### Identifying Tissue Resistivities Based on MRI Data

A relationship of tissue resistivity to the MRI pixel intensity can be expressed by the formula:

R(v)=K(1−v)^{e}+D

where R=Resistivity; v=Normalized value of MRI data; K=Multiplier value; E=Exponent; and D=Density value.

The v value can be an intensity value of either a simple MRI image or a combined image of multiple MRI images or multiple types of MRI images, normalized by the maximum possible value of the MRI data values or combined values. For example, when each pixel or voxel of the MRI data is represented by an 8 bit data, the v value for each pixel or voxel is ([the intensity value from the combined image]/[the maximum possible value from an 8 bit data (i.e., 255)]). Therefore, v has a value between zero and one.

For the interleaved image of T1, T2, and PD MRI images, constants for Equation 2 may include K=16000, E=4 and D=65. Thus, for the interleave image, a region corresponding to a v value of 1 (e.g., cerebrospinal fluid) has an R value of 65 (Ohm-cm).

In some embodiments, the equation described above is used to calculate the tissue resistivity when v has a value larger than 0.02. When v has a value that is equal to or smaller than 0.02, a predefined resistivity (e.g., 5 M ohm-cm) is used.

It is understood that persons having ordinary skill in the art recognize the reciprocal relationship between the resistivity and the conductivity. Therefore, the equation described above (or a reciprocal or multiplicative inverse function thereof) may be used in determining the tissue conductivity.

In addition, anisotropies/directionalities can be inferred from the anatomy or determined based on the MRI data, or a combination thereof. A direct determination is accomplished by diffusion tensor MRI (DT-MRI, or DTI). The indirect determination is accomplished by inferring the direction of fibers, specifically nerve fibers, by the general anatomy. DT-MRI data are sometimes called anisotrophic MRIs. In some embodiments, the anisotropies are added to the combined image to obtain anisotropic tissue properties (e.g., anisotropic electrical properties).

### Computer Systems and Methods

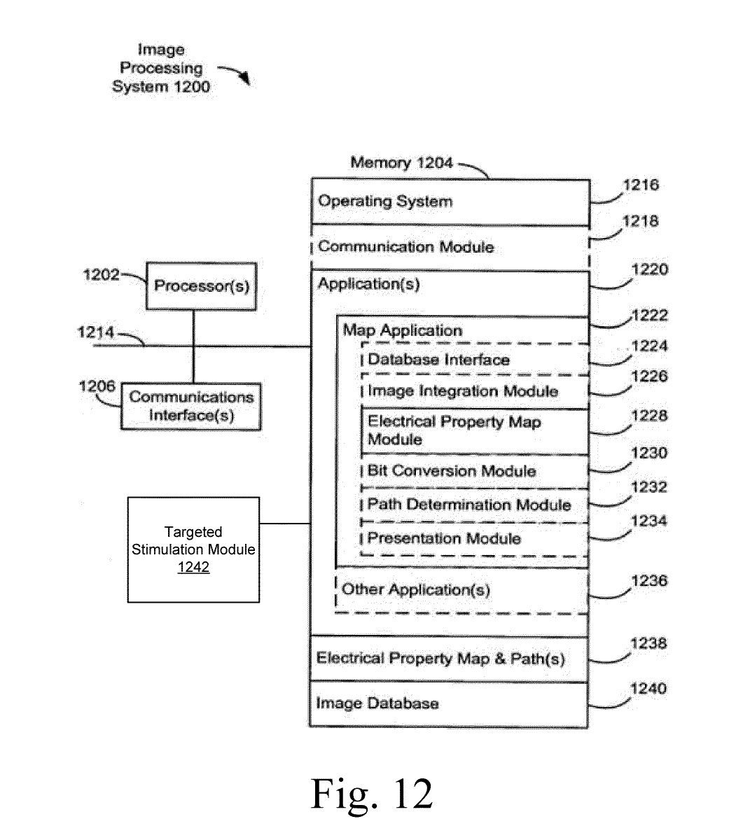

FIG. 12 is a block diagram illustrating an image processing system 1200 for processing multiple images in accordance with some embodiments. The image processing system 1200 typically includes one or more processors (CPUs) 1202, memory 1204, one or more network or other communications interfaces 1206, and one or more communication buses 1214 for interconnecting these components. In some embodiments, the communication buses 1214 include circuitry (sometimes called a chipset) that interconnects and controls communications between system components. In some other embodiments, the image processing system 1200 includes a user interface (not shown) (e.g., a user interface having a display device, a keyboard, and a mouse or other pointing device).

Memory 1204 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 1204 may optionally include one or more storage devices remotely located from the CPU(s) 1202. Memory 1204, or alternately the non-volatile memory device(s) within memory 1204, comprises a non-transitory computer readable storage medium. In some embodiments, memory 1204 or the computer readable storage medium of memory 1204 stores the following programs, modules and data structures, or a subset thereof:
Operating System 1216 that includes procedures for handling various basic system services and for performing hardware dependent tasks;
Network Communication Module (or instructions) 1218 that is used for connecting the image processing system 1200 to other computers via the one or more communications interfaces 1206 and one or more communications networks, such as the Internet, other wide area networks, local area networks, metropolitan area networks, and so on;
Application(s) 1220 that include one or more programs for execution by the one or more processors 1202;
Electrical Property Map & Path(s) 1238 that include one or more electrical property maps of one or more biological structures and one or more paths within the one or more electrical property mpas (e.g., current path); and
Image Database 1240 that includes multiple images for processing by the one or more processors 1202 and one or more combined images.

The Application(s) 1220 includes a map application 1222 include the following interfaces, modules, or a subset thereof:
Database Interface 1224 that assists retrieving, storing, or updating data in the Image Database 1238;
Image Integration Module 1226 that generates combined images from multiple images;
Electrical Property Map Module 1228 that generates an electrical property map;
Bit Conversion Module 1230 that converts M-bit data to N-bit data, where N is distinct from M (e.g., conversion from 12-bit data to 8-bit data);
Path Determination Module 1232 that identifies a current path in accordance with the electrical property map; and
Presentation module 220 that formats results from the Electrical Property Map Module 1228 and/or the Path Determination Module 1232 for display.

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, memory 1204 may store a subset of the modules and applications identified above. Furthermore, memory 1204 may store additional modules, applications, and data structures not described above.

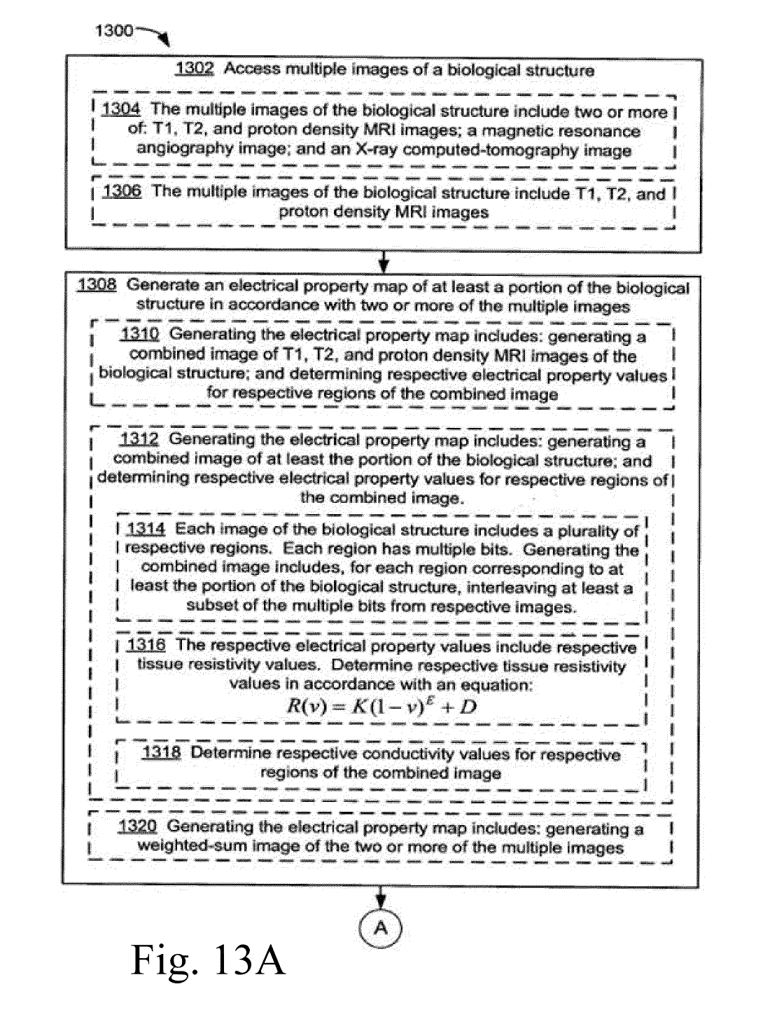

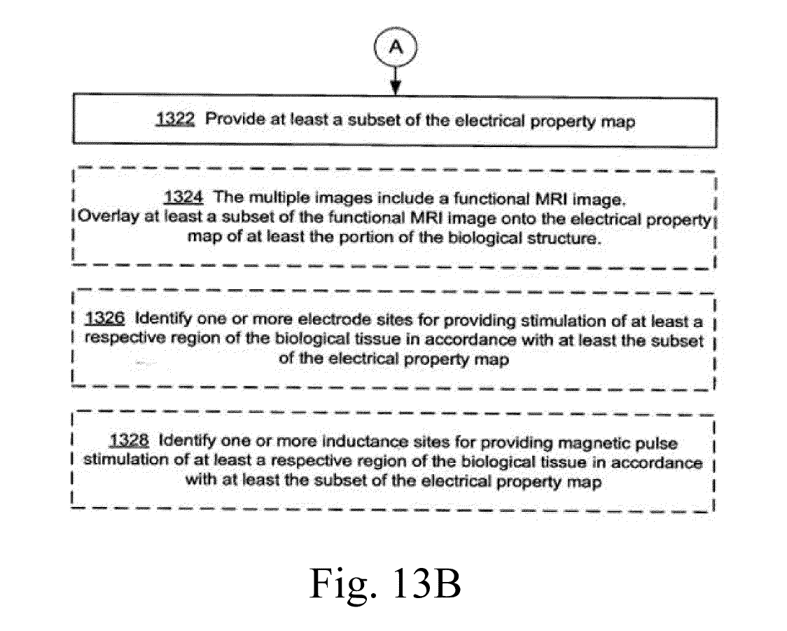

FIGS. 13A and 13B are flow diagrams illustrating a method 1300 for generating an electrical property map in accordance with some embodiments. The method 1300 may be performed at a computer system (e.g., the image processing system 1200) having one or more processors and memory storing one or more programs for execution by the one or more processors.

In one embodiment, the computer system accesses (1302) multiple images of a biological structure (e.g., T1, T2, and PD MRI images shown in FIG. 8). For example, the multiple images may be stored in the image database 1240 (FIG. 12). Alternatively, the computer system may access the multiple images stored in a remote computer system (e.g., an MRI instrument located remotely or a computer system coupled with the remotely-located MRI instrument).

In some embodiments, the multiple images of the biological structure include (1304) two or more of: T1, T2, and proton density MRI images; a magnetic resonance angiography image; and an X-ray computed-tomography image.

In some embodiments, the multiple images of the biological structure include (1306): T1, T2, and proton density MRI images (e.g., see FIG. 8).

In some embodiments, the computer system generates (1308) an electrical property map of at least a portion of the biological structure in accordance with two or more of the multiple images. For example, the combined image shown in FIG. 9 serves as an electrical property map of a slice of a brain. Such electrical property map may represent a conductivity or resistivity of tissue at each portion of the biological structure shown in the electrical property map. In some cases, the electrical property map may represent a polarity of various cells in each portion of the biological structure shown in the electrical property map.

In some embodiments, generating the electrical property map includes (1310): generating a combined image of T1, T2, and proton density MRI images of the biological structure (e.g., FIG. 9); and determining respective electrical property values for respective regions of the combined image (e.g., determining resistivity for each pixel or voxel of the combined image). In some embodiments, the computer system converts pixel intensities to obtain electrical property values.

In some embodiments, generating the electrical property map includes (1312): generating a combined image of at least the portion of the biological structure; and determining respective electrical property values for respective regions of the combined image. For example, the computer system converts pixel intensities to electrical property values (e.g., using the Equation 2).

In some embodiments, each image of the biological structure includes (1314) a plurality of respective regions (e.g., pixels or voxels), and each region has an intensity value represented with multiple data bits (e.g., 8-bits). Generating the combined image includes, for each region corresponding to at least the portion of the biological structure, interleaving at least a subset of the multiple bits from respective images (e.g., see FIG. 11).

In some embodiments, the respective electrical property values include (1316) respective tissue resistivity values. The computer system, as described above, may determine respective tissue resistivity values in accordance with an equation:

R(v)=K(1−V)^{e}+D

where R is a respective tissue resistivity value, K is a multiplier value, v is a respective normalized numeric value stored in the combined image, E is an exponent, and D is a density value.

In some embodiments, the computer system determines (1318) respective conductivity values for respective regions of the combined image. For example, the computer system may determine respective resistivity values and then calculate reciprocal values of the respective resistivity values.

In some embodiments, generating the electrical property map includes (1320) generating a weighted-sum image of the two or more of the multiple images. For example, the computer system may determine the intensity of each pixel in a combined image by using Equation 1 described above. In some embodiments, the weights used in generating the weighted-sum image are determined in accordance with an approximation method (e.g., least-squares).

In some embodiments, the electrical property map includes a plurality of respective regions (e.g., pixels or voxels), and each region has an electrical property value that is isotropic (e.g., a conductivity or resistivity that is orientation-independent).

In some embodiments, generating the electrical property map includes obtaining a plurality of anisotropic electrical property values, and adjusting the electrical property map in accordance with the plurality of anisotropic electrical property values (e.g., adding respective anisotropic electrical property values to corresponding isotropic electrical property values in the electrical property map). Obtaining the plurality of anisotropic electrical property values typically includes processing one or more diffusion-weighted images of the biological structure. For example, the one or more diffusion-weighted images of the biological structure may be analyzed (e.g., using the diffusion tensor analysis method or the Q-ball analysis method) to obtain the plurality of anisotropic electrical property values.

The computer system, in one embodiment, provides (1322, FIG. 13B) at least a subset of the electrical property map. For example, the computer system displays at least the subset of the electrical property map. Additionally or alternatively, the computer system transmits at least the subset of the electrical property map to a second computer system typically located remotely from the computer system. The second computer system may be located at a hospital or in a doctor's office so that a physician or a surgeon may review the electrical property map.

In some embodiments, the multiple images include (1324) a functional MRI image. The computer system overlays (1324) at least a subset of the functional MRI image onto the electrical property map of at least the portion of the biological structure.

In some embodiments, the computer system identifies (1326) one or more electrode sites for providing stimulation of at least a respective region of the biological tissue in accordance with at least the subset of the electrical property map. The details of selection of one or more electrode sites are described in detail with respect to the section entitled “Stimulation Site Selection.”

In some embodiments, the computer system identifies (1328) one or more inductance sites for providing magnetic pulse stimulation of at least a respective region of the biological tissue in accordance with at least the subset of the electrical property map.

### Individual Differences and Developmental Variations

Bone is generally the highest resistivity tissue in the body thus making the skull a significant barrier to injection currents. There are also considerable variations in skull thickness and density between sites within and between individuals. The cranial sutures, penetrating vessels and individual anomalies provide low resistivity paths through the skull that are important sources of individual variation.

Developmentally, the presence of highly vascularized fontanel in young children provides a path for current through the skull, because of the fontanel's much lower resistivity (scalp: 230 ohm-cm; blood: 160 ohm-cm; bone 7560 ohm-cm) compared with the surrounding bone. These fontanels are substantially closed by 1.5 years to form the sutures present in the adult skull. The sutures remain open for some time in many adults, and do not close at all in some aged individuals, although in others they close completely. By adjusting for these differences rather than simply increasing the current, the currents needed to stimulate the brain of an individual can be reduced.

FIGS. 1A and 1B were introduced earlier. FIG. 1A shows MEPs evoked by transcranial stimulation in a 14 year old scoliosis patient. The electrode positions were approximately at C1 and C2 (10-20 system), with anodal stimulation applied at C2 (50V). The largest amplitude MEPs were evoked from muscles of the left foot (abductor hallucis) and leg (anterior tibialis), although smaller responses from the abductor hallucis muscle on the right side was also noted. No responses were recorded in the abductor pollicis brevis muscles of either hand. These relatively low current responses were obtained by slight adjustments in electrode locations. Similar adjustments varying from patient to patient may be used to optimize MEP signals.

In alternative embodiments, it is possible to reduce the level of stimulation for intraoperative monitoring and improve our understanding of what is occurring with tcMEP. In some embodiments, however, significant further improvement is achieved. Additional improvements are provided in the model by: 1) utilizing a three-dimensional GETs model; 2) improving the detail in the images to account for blood vessels, finer nerve tracks and bone anomalies; 3) adding into the model the effects of capacitance found at tissue boundaries; 4) verifying the model with direct brain measurements; or 5) by applying findings to the motor cortex in refractory Parkinsonism patients, or combinations thereof.

In one embodiment, GETs models are provided in 3-D, and finer detail is applied to the images, while effects of capacitance are added. As a result, the electrical property (or properties) represented by the electrical property map changes from resistivity to impedance.

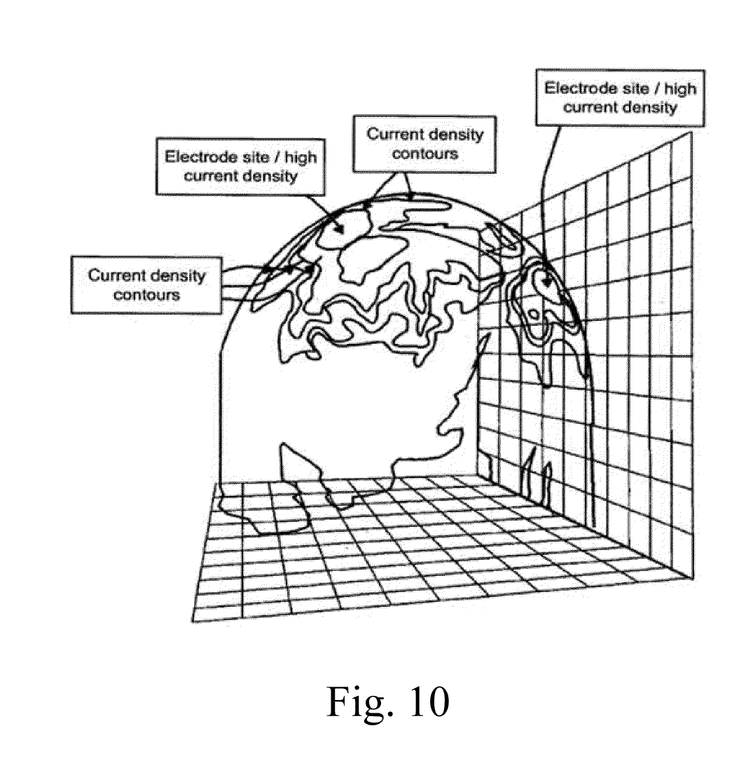

FIG. 10 illustrates three-dimensional modeling of current densities applied to a human brain coupled with two electrodes. FIG. 10 shows contours of constant current densities. FIG. 10 illustrates the high current density around the electrodes and changing current densities along any current path that traverses multiple tissues. In one embodiment, the images are segmented, a FE mesh is generated, and then the analysis is performed for isotropic models and/or anisotropic models with and without capacitance. In some embodiments, capacitance is an important factor as membrane capacitance at tissue boundaries as well as a significant factor in determining stimulus tissue penetration.

### Segmentation

Segmentation, or the outlining, identifying, ascribing and/or assigning of resistivity values to MRI slices in 3-D, can be a difficult and arduous task. The effort involved may be significantly reduced by using commercial automated tissue analysis algorithms and services. For example Neuroalyse, Inc (Quebec, Canada), may be selected to perform such analysis. This system can perform more than 90% of the tissue segmentation and leave blank the areas of the tissue that the software is unable to resolve or where it is preferred to more particularly work with these areas. This automated segmentation is particularly advantageous as new MRI images have 2 mm thicknesses and record in three planes. The results are checked and any blank areas filled in by hand or other precision automation, or otherwise. Tissue resistivities are assigned, except tissue slices may be finer and values may be included for blood vessels and skull sutures. Resulting 2-D sliced images are then interleaved into a three 3-D model. A final 3-D segmentation and meshing may be performed using AMIRA (Mercury Computer Systems, Berlin, Germany) and the resulting 3-D models generated may be imported into FEMLAB (Comsol, Burlington Mass.) for FE calculation.

The 3-D images, with identified motor cortex, may be analyzed using the FE method. To identify the best sites for stimulation, an additional analysis may be performed by iteratively moving representative paired electrode locations across the scalp in the FE model and evaluating effects (e.g., the current density at the target site, such as motor cortex, and/or other sites). This targeting may be performed by having the computer systematically select and test for the highest current density at the target site for each of the locations of the traditional 10-20 system for electrode placements as current injection and extraction sites with a constant current pulse. In addition to the traditional 10-20 system, other sites that may be considered or selected may include eye lids, auditory canals and nasal passages as these additional locations represent avenues for bypassing the high resistivity of the skull bone. After the computer has grossly identified a pair of stimulation and extraction sites, the model may be refined by testing in one centimeter increments around selected sites of the 10-20 system.

These predicted “best fit” locations may then be tested against the two “standard” locations most commonly presented in the current literature (C3-C4 and Cz′-FPz of the 10-20 system). This 3-D effort provides an advantageously sophisticated model, although verification and human testing may still be used as well.

In a further embodiment, the technique includes adding CT scans to MRI images, and/or applying the model to spinal surgery patients. MRI is effective at imaging soft tissue, but is less effective at imaging bones, because MRI is effective at imaging water molecules within the target tissues. The bony skull is the highest resistivity tissue in the head and a significant barrier for electric current passing into the brain. Our modeling has compensated for this by assuming that dark regions between the brain and the scalp are bony structures. This has the advantage of requiring a single scan of a patient, as long as the quality remains high. Alternatively, MRI images may be collected in three axes (axial, coronal, and sagital), and CT images may be scanned and retroactively adjusted to match the three axes of the MRI scans. The two sets of images may then be digitally co-registered and combined as described above.

### Direct Measurement

Currents may be directly measured in the cerebral ventricle of patients who are about to have a ventricular drain placed in their brain for elective shunt placement for hydrocephalus. In this clinical procedure, a small craniotomy is performed, the dura is then opened, and one end of a silastic tube is placed through the brain and into the ventricle for the purpose of draining excess cerebrospinal fluid. This sylastic tube is filled with saline or cerebrospinal fluid to avoid bubbles and used as a drain. Thus, a saline filled tube can act as a recording electrode placed in the ventricle and passing through brain tissues. Record from this tube may be performed by inserting a platinum/iridium probe in the distal end of the tube and connecting the probe to a recording oscilloscope. After the oscilloscope is turned on, three sets of transcranial pulses will be applied to the patient and the pulsed current measured from the ventricular space will be measured. To reach the ventricle, the tube is placed through a section of prefrontal cortex and readings are taken in this region as well. The readings for the current levels in the sampled regions may be compared to the current levels predicted by the GETs model. The sylastic ventricular drain tube itself has resistivity and capacitance properties and these may be determined and tested by placing the tube in a saline filled beaker and testing the resistivity and capacitance of the tube before it is placed in the subject's brain or added to the model.

### Biological Assay

In some embodiments, a biological assay is performed to test stimulation of the motor cortex in patients who are having elective spinal surgeries that require tcMEPs as part of their surgical monitoring procedure. Effective current levels for stimulation in clinical patients may be established in this way. Since there is variation in the fine detail location of the motor cortex between individuals, it is advantageous to determine with precision the location of the target muscle as represented in the cortex.

Motor cortex localization may be determined by functional MRI (fMRI). The fMRI may be performed with the subject instructed to move his or her thumb (the abductor pollicis brevis muscle) to obtain precision location information of that muscle's representation in the motor cortex while the fMRI is being performed. The resulting imaged location can then be the target location for modeling of stimulation. The subject's MRI (and/or CT) image is segmented as described. The subject's data are then received for GETs modeling for stimulation.

### Stimulation Site Selection

The best location for stimulating electrodes for targeting an identified motor cortex may be selected by the following method. First, the target site is identified. The computer may be programmed to systematically select and test for current density at the target site for each of the locations of the traditional 10-20 system for electrode placements on the head as current injection and extraction sites. In addition to the traditional 10-20 system sites, the eye lids, auditory canals and the nasal passage are typically added, as they represent relevant avenues for bypassing the high resistivity of the skull. After the computer has grossly identified a pair of stimulation and extraction sites, the model may be refined in one centimeter increments around estimated sites. The computer may iterate moving the stimulation sites until new optimized sites are selected for use. The criteria the computer will use for target site evaluation may be the highest current achieved when a 10 Volt constant current square wave signal is modeled. The selected stimulation model is also examined for potential stray currents. In some embodiments, for safety, the selected stimulation model is eliminated if it is judged to affect an area that might produce side effects. In some embodiments, the electrode sites are identified as a pair (i.e., two electrode sites are identified). In some other embodiments, three or more electrode sites are identified as a set.

It should be appreciated that with the method described above, the electrode sites can be selected solely from homogeneous or isotropic tissue properties (e.g., resistivity or conductivity values), without using anisotropic properties. Therefore, the method described above eliminates the need for any anisotropic measurement or a database storing anisotropic property values.

Conversely, one or more target sites may be determined from a given set of electrode sites. First, one or more sets of potential electrode sites are identified either manually or using predefined coordinates, such as the 10-20 system. The computer system may use the electrical property map and the FE method to identify the current path for each set of electrode sites. In some embodiments, the computer system also determines a site within the biological tissue that has the highest current density. In some embodiments, the current density at one or more target sites may be determined from the given set of electrode sites.

### tcMEP Recording Conditions

Anesthesia levels, blood pressure, and body temperature is generally kept constant during the tcMEP recording. No muscle relaxants are used for the preferred procedure, except during intubation. The low current levels allow stimuli to be presented through subdermal electrodes. During a patient's surgery, a patient may receive total intravenous anesthesia (TIVA) with propofol and narcotics to negate the inhibiting effect that traditional inhalation agents have on the motor cortex. These procedures are generally several hours long and testing can be done during a stable anesthetic regimen. The motor responses may be recorded from subdermal needle electrodes placed in the target muscle and recorded on a Cadwell Cascade intraoperative monitoring machine. Stimuli may be short duration square wave pulses presented through a constant current stimulator. The exact duration and intensity may be determined by the impedance properties predicted by the modeling.

The stimulus parameters may be identical between groups with a train of 6 square wave 100 μsec pulses with a fixed inter-stimulus interval (ISI) and constant voltage. A minimum voltage and location may be determined by the model or the traditional sites found in the literature. The outcome variable may be the amplitude and duration of response as a reflection of the number of neurons activated in the fMRI identified loci of the motor cortex.

### Electrode Within or Through the Skull

The skin is a low resistance medium (approximately 230 ohms per cm) and the skull is very high resistance (approximately 1600 ohms per cm). When two or more electrodes are placed on the scalp and electrical energy is passed between them, most of the energy applied passes through the skin and relatively little goes into the brain. Thus the pain that is often felt when electrical current is applied to the head is really the result the electrical current that is passing through pain receptors in the scalp, and not to the stimulus that is reaching the brain. This can tend to limit amounts of electrical stimulus that can be applied to patients for therapy. This shunting of electrical energy though the scalp can be significantly reduced by placing electrodes within or through the skull and insulating the electrode from the scalp. In this manner electrical energy is directed away from the scalp and towards the brain.

### Methods of Using the Electrical Property Map

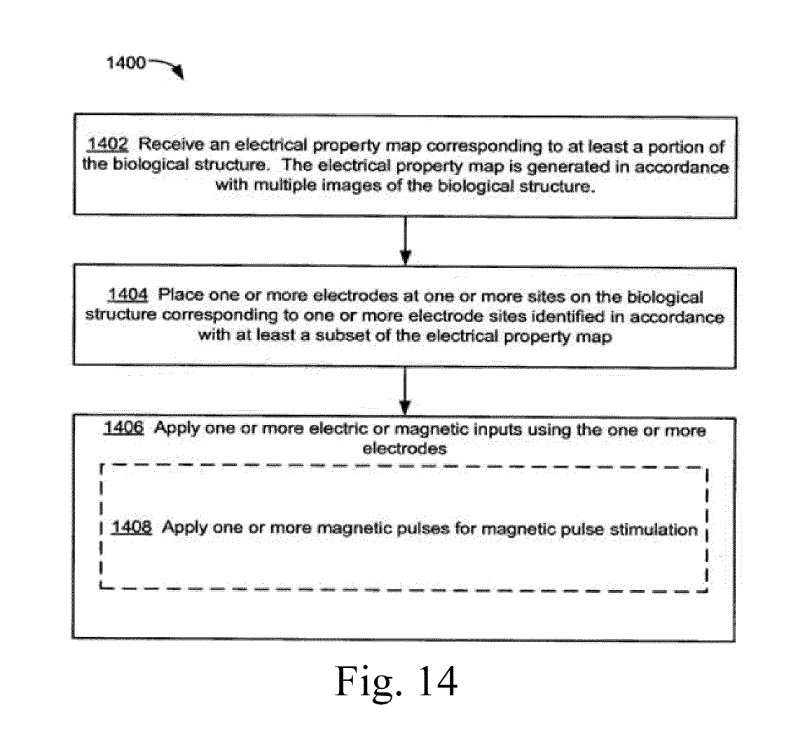

FIG. 14 is a flow diagram illustrating a method 1400 for providing electrical or magnetic stimulation of a biological structure in accordance with some embodiments. The method, in one embodiment, includes (1402) receiving an electrical property map corresponding to at least a portion of the biological structure. The electrical property map is generated in accordance with multiple images of the biological structure (e.g., T1, T2, and PD MRI images of the biological structure, such as brain).

The method, in one embodiment, includes (1404) placing one or more electrodes at one or more sites on the biological structures corresponding to one or more electrode sites identified in accordance with at least a subset of the electrical property map. In some embodiments, the one or more electrode sites are selected based on one or more simulation results indicating that the current density at a target site (e.g., a portion of the biological structure where application of electrical current is desired) meets a predefined effective current threshold. In some embodiments, the one or more electrode sites are selected based on one or more simulation results indicating that the current density at one or more sites other than the target site does not exceed a predefined safety threshold. For tcMEP monitoring, the one or more electrode sites are located on the scalp (e.g., electrode sites in FIG. 10).

The method, in one embodiment, includes (1406) applying one or more electric or magnetic inputs using the one or more electrodes (e.g., applying electrical input of a predefined voltage for 100 μs in trains of five pulses). In some embodiments, the method includes monitoring motor evoked potentials in response to the one or more electric or magnetic inputs. In some embodiments, the one or more electric or magnetic inputs (e.g., current and/or voltage) are selected based on one or more simulation results indicating that the current density at the target site meets the predefined effective current threshold. In some embodiments, the one or more electric or magnetic inputs are selected based on one or more simulation results indicating that the current density at one or more sites other than the target site does not exceed a predefined safety threshold.

In some embodiments, the method includes (1408) applying one or more magnetic pulses for magnetic pulse stimulation.

When one or more electrode sites that can more effectively transmit the electrical or magnetic energy to the target site are selected with the electrical property map, a desired level of the current density can be achieved at the target site with electrical currents lower than those used in conventional methods. This is beneficial in many applications. For example, reduced currents may be used to deliver brain stimulation in awake patient populations.

As explained above, electrical property maps can be used for monitoring purposes (e.g., tcMEP monitoring). In some cases, transcranial electrical stimulation may be used in awake patients, as long as discomfort and pain involved are low enough, i.e., when current levels applied across the scalp are low enough as in accordance with some embodiments. For example, the use of the electrical property maps permits reduction of currents to less than 20 mA (at a constant voltage), which may be applied to awake patients for tcMEP monitoring during surgery.

In addition, electrical property maps can be used for therapeutic purposes as well. For example, the reduced currents may be used for treating patients with refractory depression, epilepsy and chronic pain. In some embodiments, such patients may be treated with the electrical current while they are awake, because the applied currents are low.

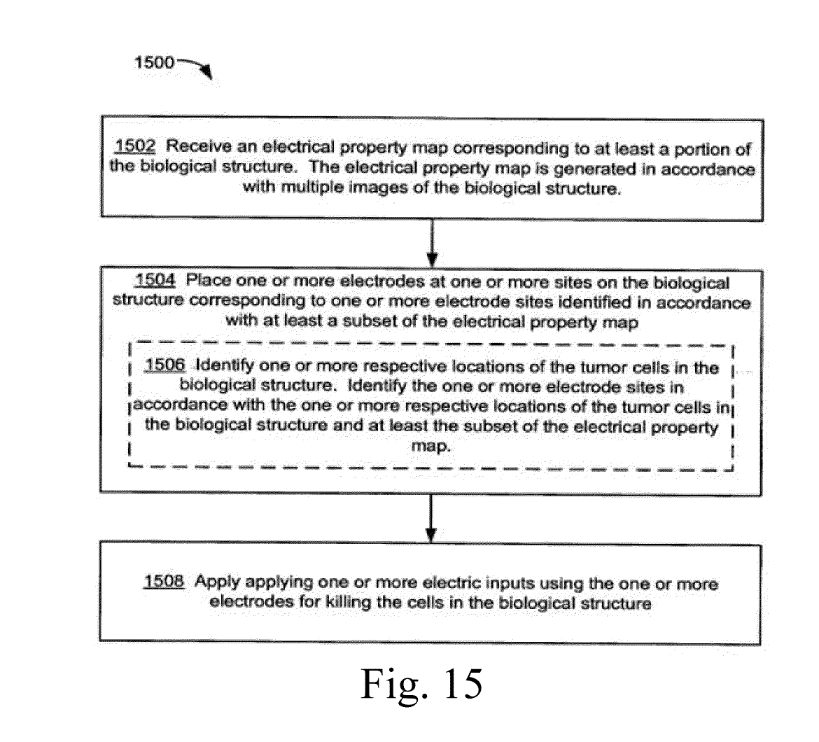

FIG. 15 is a flow diagram illustrating a method 1500 for killing cells in accordance with some embodiments. The cells typically include pathological cells, such as tumor cells.

The method, in one embodiment, includes (1502) receiving an electrical property map corresponding to at least a portion of a biological structure. The electrical property map is generated in accordance with multiple images of the biological structure.

The method, in one embodiment, includes (1504) placing one or more electrodes at one or more sites on the biological structure corresponding to one or more electrode sites identified in accordance with at least a subset of the electrical property map. Although many embodiments are described herein with respect to a brain, it should be noted that the methods and systems described herein are not limited to application to a brain or head region. For example, the one or more electrode sites may be located on any biological structure.

In some embodiments, the method includes (1506) identifying one or more respective locations of the tumor cells in the biological structure; and identifying the one or more electrode sites in accordance with the one or more respective locations of the tumor cells in the biological structure and at least the subset of the electrical property map. For example, the one or more respective locations of the tumor cells may be determined from one or more MRI images (e.g., the multiple MRI images used for generating a combined image), CT scans, and/or by means of other radiological or pathological methods and tools (e.g., ultrasound scans, PET scans, histology, etc.).

The method, in certain embodiments, includes (1508) applying one or more electric inputs using the one or more electrodes for killing the cells in the biological structure. The one or more electric inputs may include an electrical input sufficient to kill the cells. Typically, the cells located in the current patch will be killed or damaged. Alternatively, the method includes applying one or more electric inputs using the one or more electrodes for arresting proliferation of tumor cells.

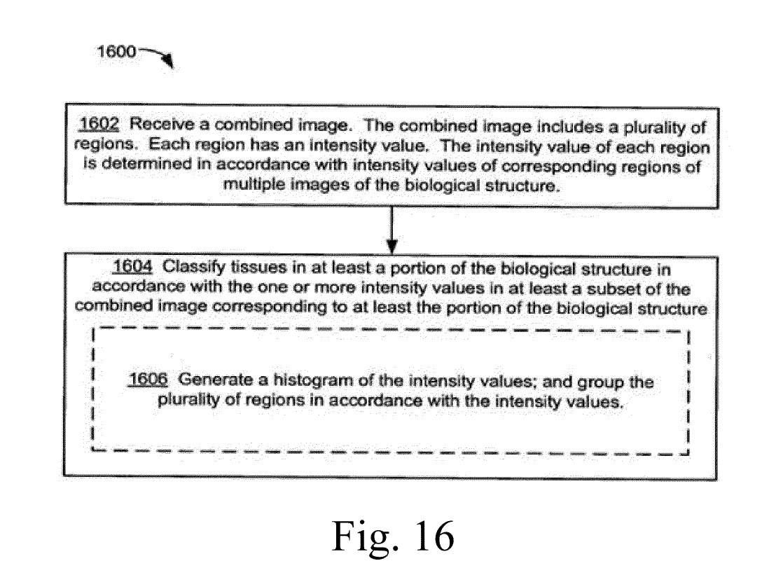

FIG. 16 is a flow diagram illustrating a method 1600 for classifying tissues in accordance with some embodiments. The method 1600, in one embodiment, is performed at a computer system having one or more processors and memory storing one or more programs for execution by the one or more processors for performing tissue classification (e.g., the image processing system 1200, FIG. 12). As used herein, the terms “tissue classification” and “classifying tissue” refer to grouping tissues of a similar property together. Tissue classification does not necessarily involve identifying a tissue based on the tissue property. In other words, tissue classification may be used to determine that two separate portions of an image correspond to a same type of tissue, but tissue classification does not necessarily identify the type of tissue (e.g., white matter v. gray matter) that those portions of the image correspond to. However, additional steps (e.g., histology examination) may be followed to identify tissue in each group, thereby correlating the tissue property with a particular group of tissues (e.g., tumor cells).

The computer system, in one embodiment, receives (1602) a combined image (e.g., the combined image in FIG. 9). The combined image includes a plurality of regions, and each region has an intensity value. The intensity value of each region is determined in accordance with intensity values of corresponding regions of multiple images of the biological structure.

The computer system, in one embodiment, classifies (1604) tissues in at least a portion of the biological structure in accordance with the one or more intensity values in at least a subset of the combined image corresponding to at least the portion of the biological structure. For example, the computer system groups the tissues shown in the combined image of FIG. 9 based on the intensity values.

In some embodiments, the computer system generates (1606) a histogram of the intensity values, and grouping the plurality of regions in accordance with the intensity values. For example, FIG. 9 shows that each group of regions is represented as one or more respective peaks in the histogram.

### Targeted Electrical Stimulation Using Clustering

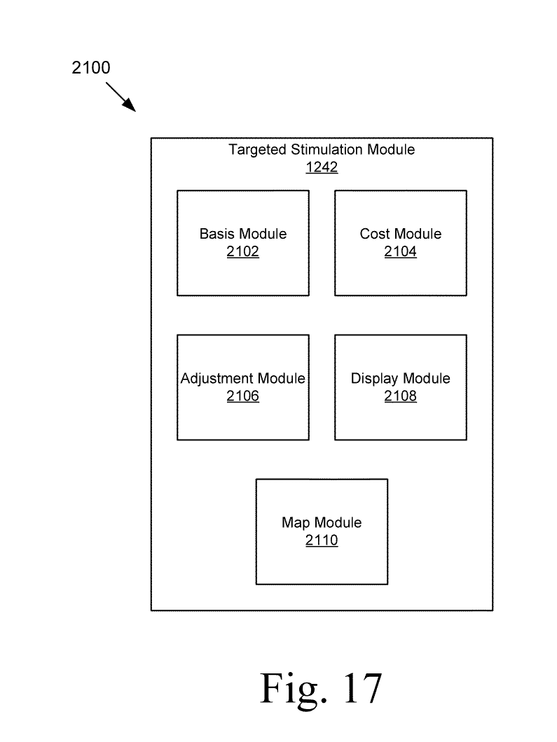

FIG. 17 depicts one embodiment of a module 2100 for targeted electrical stimulation. In one embodiment, the module 2100 includes an embodiment of a targeted stimulation module 1242. The targeted simulation module 1242, in various embodiments, includes one or more of a basis module 2102, a cost module 2104, an adjustment module 2106, a display module 2108, and a map module 2110, which are described in more detail below.

The targeted stimulation module 1242, in one embodiment, may be located on a computing device, such as a desktop computer, a laptop computer, a tablet computer, a smart phone, a server, or the like (e.g., such as the computing device described above with reference to FIG. 12). In various embodiments, the targeted stimulation module 1242 may be embodied as hardware, software, or some combination of hardware and software. In one embodiment, the targeted stimulation module 1242 may comprise executable program code stored on a non-transitory computer readable storage medium for execution on a processor of a computing device. For example, the targeted stimulation module 1242 may be embodied as executable program code executing on a desktop computer, a smart phone, etc. In such an embodiment, the various modules that perform the operations of the targeted stimulation module 1242 may be located on one or more computing devices.

The targeted stimulation module 1242, in various embodiments described below, is configured to simulate electrical stimulation of a target area to determine an optimal configuration of electrodes that maximizes the stimulation to one or more target areas while minimizing the stimulation of non-target areas not intended to be stimulated by the electrodes. To achieve this, the targeted stimulation module 1242 determines a configuration of electrodes based on a resistivity map for an individual, iteratively calculates a cost of stimulating a target area based on the configuration and adjusts the configuration of electrodes until a minimum cost threshold is satisfied, and graphically presenting the optimal electrode configuration that minimizes the cost of electrically stimulating the target area.

As used herein, the cost may describe a measurement of the effect of transmitting an electrical current to a target area based on a current configuration of electrodes. In certain embodiments, the cost is associated with maximally stimulating a target area while minimally stimulating non-target areas, which may depend on the objectives of the stimulation, e.g., the condition being treated, determined by a domain expert such as a physician, therapist, or the like. For example, a domain expert may want to maximize the electrical energy at a targeted area regardless of the electric current direction, or he/she may want to maximize electric current in a particular direction, all while avoiding stimulation of areas outside the target area.

In various embodiments, the targeted stimulation module 1242 may be embodied as a hardware appliance that can be installed or deployed in a computing device, system, network, and/or the like. In certain embodiments, the targeted stimulation module 1242 may comprise a hardware device such as a secure hardware dongle or other hardware appliance device (e.g., a set-top box, a network appliance, or the like) that attaches to another computing device, such as a laptop computer, a server, a tablet computer, a smart phone, or the like, either by a wired connection (e.g., a USB connection) or a wireless connection (e.g., Bluetooth®, Wi-Fi®, near-field communication (NFC), or the like); that attaches to an electronic display device (e.g., a television or monitor using an HDMI port, a DisplayPort port, a Mini DisplayPort port, VGA port, DVI port, or the like); and/or the like. A hardware appliance of the security module 104 may comprise a power interface, a wired and/or wireless network interface, a graphical interface that attaches to a display, and/or a semiconductor integrated circuit device as described below, configured to perform the functions described herein with regard to the targeted stimulation module 1242.

The targeted stimulation module 1242, in such an embodiment, may comprise a semiconductor integrated circuit device (e.g., one or more chips, die, or other discrete logic hardware), or the like, such as a field-programmable gate array (FPGA) or other programmable logic, firmware for an FPGA or other programmable logic, microcode for execution on a microcontroller, an application-specific integrated circuit (ASIC), a processor, a processor core, or the like. In one embodiment, the targeted stimulation module 1242 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the targeted stimulation module 1242.

The semiconductor integrated circuit device or other hardware appliance of the targeted stimulation module 1242, in certain embodiments, comprises and/or is communicatively coupled to one or more volatile memory media, which may include but is not limited to: random access memory (RAM), dynamic RAM (DRAM), cache, or the like. In one embodiment, the semiconductor integrated circuit device or other hardware appliance of the security module 104 comprises and/or is communicatively coupled to one or more non-volatile memory media, which may include but is not limited to: NAND flash memory, NOR flash memory, nano random access memory (nano RAM or NRAM), nanocrystal wire-based memory, silicon-oxide based sub-10 nanometer process memory, graphene memory, Silicon-Oxide-Nitride-Oxide-Silicon (SONOS), resistive RAM (RRAM), programmable metallization cell (PMC), conductive-bridging RAM (CBRAM), magneto-resistive RAM (MRAM), dynamic RAM (DRAM), phase change RAM (PRAM or PCM), magnetic storage media (e.g., hard disk, tape), optical storage media, or the like.

In one embodiment, the basis module 2102 is configured to determine a base configuration for each electrode of a plurality of electrodes that are configured to transmit or direct an electrical current towards a target area of a biological structure. As described above, an electrode is a conductor through which electricity enters or leaves an object, substance, or region. An electrode configuration, in one embodiment, includes a location for an electrode (e.g., a location on a person's head, chest, or the like), a current strength and/or direction generated by one or more electrodes, a total number of electrodes, and a size and/or shape of an electrode. The biological structure, for example, may include an individual's head, brain, chest, heart, lungs, etc. The target area may include at least a portion of the biological structure, such as the caudate, amygdala, or the like within a brain.

In one embodiment, the target area is a region of connected pixels or voxels in a person's electrical property map, head conductivity map, resistivity map, or the like, as described in detail above. In certain embodiments, the basis module 2102 receives input from a user that specifies a target area of the biological structure, one or more pixels/voxels that comprise the target area, or the like. In a further embodiment, a plurality of areas of the biological structure may be designated as target areas.

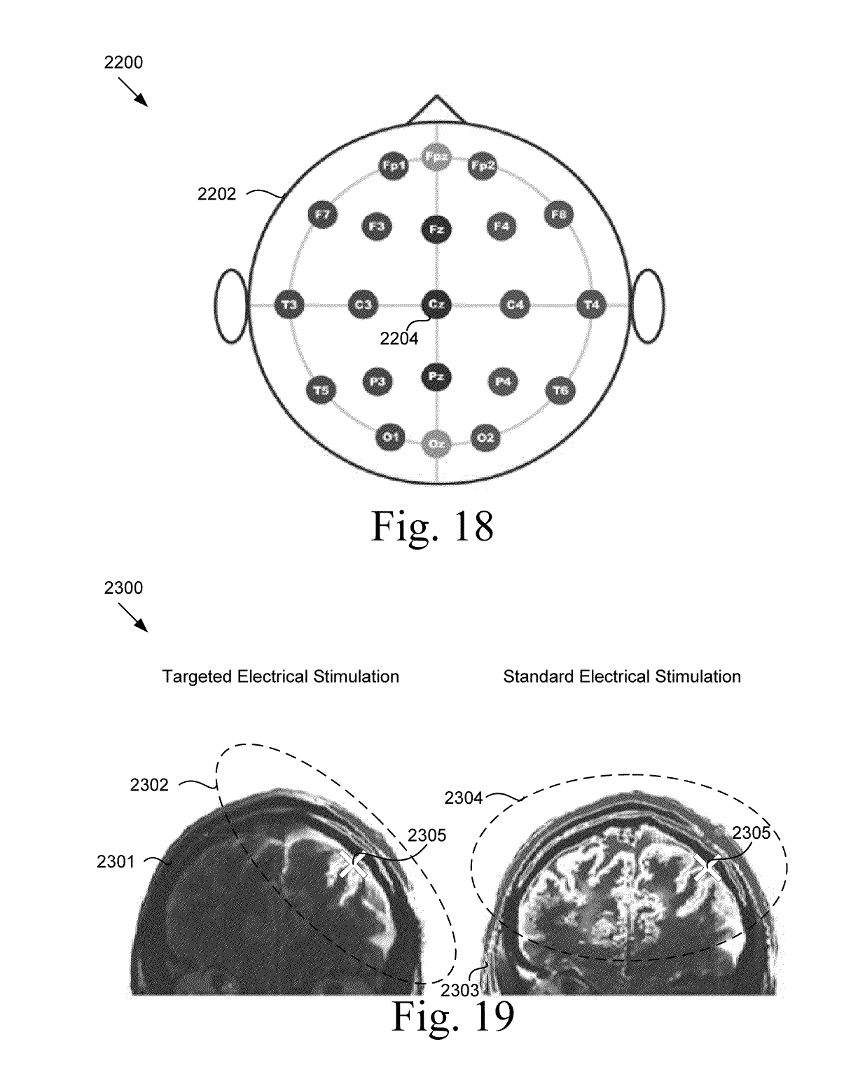

In one embodiment, the basis module 2102 determines an initial or base location for each electrode of a plurality of electrodes. The basis module 2102 may determine a base location from a predefined system for biological structure and/or the target area of the biological structure. For example, if the target area is an area within an individual's brain, the basis module 2102 may determine a base location for each electrode according to a 10-10 system, a 10-20 model for the individual's head, as shown in FIG. 18, or another predefine geographic placement of electrodes. The 10-20 model, as described above, is a predefined system to describe and apply the location of scalp electrodes 2204 in the context of an EEG test or experiment, for example, to an individual's head 2202. The 10-20 system is based on the relationship between the location of an electrode and the underlying area of cerebral cortex. The “10” and “20” refer to the fact that the actual distances between adjacent electrodes are either 10% or 20% of the total front-back or right-left distance of the skull.

In one embodiment, the basis module 2102 determines a configuration for each electrode of the plurality of electrodes based on the location of the target area and/or a resistivity map of the target area and/or the area surrounding the target area. For example, the basis module 2102 may determine a location on a person's scalp for an electrode, a size of the electrode, a shape of the electrode, a current strength for the electrode, and a total number of electrodes to be placed on or in a person's head to stimulate a target area within the person's brain based on a resistivity map generated specifically for the person.

As described above, and explained in greater detail below with reference to the map module 2110, the map module 2110 may generate a resistivity map (or an electrical property map where pixels/voxels represent electrical resistivity for different biological material types) for a biological structure of an individual. Based on the resistivity map, the basis module 2102, and/or the adjustment module 2106 described below, may determine a configuration for the electrodes that maximizes stimulation of the target area of the biological structure while minimizing stimulation of surrounding or non-target areas of the biological structure.

Furthermore, the basis module 2102 and/or the adjustment module 2106 may determine a configuration for each of the electrodes using the resistivity map such that the electrical current is clustered at or near the target area based on the various resistivity values of the material comprising the biological structure. For example, as shown in FIG. 7A, the biological structure may be comprised of various tissues that each have different electrical resistivity properties. Some tissues 702 may be highly resistant to electrical current, while others 704 may have low resistance to electrical current, and the rest 706 may have some kind of medium resistance to electrical current. Accordingly, based on the resistivity map, the basis module 2102 and/or the adjustment module 2106 may determine one or more “hot spots” 708 where the electrical current tends to cluster or focus, and based on the “hot spots” the basis module 2102 and/or the adjustment module 2106 can determine a configuration of the electrodes that may focus or cluster electrical current towards the target area while minimizing negative side effects of stimulating other non-target areas of the biological structure, e.g., minimizing the cost of the electrical stimulation.

The basis module 2102, in a further embodiment, determines a common ground for one or more electrodes. The common ground, in one embodiment, comprises one or more fixed locations for electrodes that absorb excessive current from and/or provide deficit current to the target area, e.g., a wrist, or the like. In some embodiments, the fixed locations depend on the configuration of electrodes that the basis module 2102 determines. For example, the basis module 2102 may determine fixed locations for the common ground electrodes based on an initial configuration of electrodes configured to electrically stimulate the target area. Thereafter, the basis module 2102 (or the adjustment module 2106 described below) may modify the initial configuration of electrodes configured to electrically stimulate the target area. Based on the modified configuration, the basis module 2102 may also modify the fixed locations for the common ground electrodes. In certain embodiments, basis module 2102 determines the fixed locations for the common ground according to a FEM model. In a further embodiment, the common ground acts as a voltage reference for the electrical stimulation of the target area.

The cost module 2104, in one embodiment, calculates a cost associated with a simulation of providing an electrical current to the target area according to the configuration for each of the electrodes. The cost, in certain embodiments, is a measurement, such as a total amount of current, current density, or the like, of the effect of transmitting an electrical current to the target area and other non-target areas, based on a current configuration of electrodes. The cost module 2104 may calculate the cost of the targeted stimulation as a function of the location of the electrodes, the current strength of the electrodes, the sizes of the electrodes, the shapes of the electrodes, the number of electrodes, and/or the like. In various embodiments, the cost module 2104 calculates the cost according to a predefined cost function, described in greater detail below, which may incorporate one or more variables, conditions, constraints, or the like, associated with providing an electrical current to a target area of the biological structure.

In one embodiment, the cost module 2104 calculates the cost as a function of a cost associated with the target area and a cost associated with one or more areas outside the target area, e.g., avoided areas. For example, let the targeted area be denoted by Ω_{T} and the avoided area by Ω_{A}. As described above, the targeted and avoided areas may be indicated, specified, or otherwise provided by a domain expert. Other areas outside of the target area and the avoided area may be denoted by Ω_{D}.

Continuing with the current example, let E_{T} be the cost function associated with achieving objectives on the target area Ω_{T} and E_{D} be the cost function associated with achieving objectives on Ω_{D}. In general, a reduction of the cost E_{T} may correspond to a better compliance of the domain expert's prescription in the target area Ω_{T}, while the reduction of the cost E_{D} may correspond to a weaker stimulation in Ω_{D}. A weighted combination of αE_{T}+βE_{D}, where α+β=1, may be understood as a compromise of the two goals of stimulating particular target area(s) of the biological structure, e.g., the brain, and reducing stimulation to other areas to achieve targeted stimulation.

The cost module 2104, in certain embodiments, calculates the cost of stimulating the target area subject to one or more constraints. For example, the cost module 2104 may calculate the cost of stimulating a target area within a brain subject to one or more safety constraints for the brain. The constraints may include an upper/lower bound on electric current going through (in or out) any individual electrodes, an upper/lower bound on total electric current going through all the electrodes, an upper/lower bound on current density in a particular area of the biological structure (e.g., an area designated by Ω_{A}, or the like), and/or the like. Let the collection of constraints applicable to a targeted stimulation model be denoted by C={C₁, . . . , Cₙ}.

Continuing with the example above, let Γ be the collection of all possible electrode configurations—configurations that include varying the total number of electrodes, the location of the electrodes, the electrical current strength, and the sizes and/or shapes of the electrodes. E_{T}, E_{D}, and C, above, may all be functions defined on Γ. The targeted stimulation approach may be formulated as:

minimize_{γ} αE_{T}(γ)+βE_{D}(γ) subject to Cᵢ(γ), i=1, . . . , n.

In certain embodiments, the above formulation is too high-dimensional and may be computationally unfeasible. Moreover, it may be more practical to use as few electrodes as possible. Accordingly, Γ may be initially approximated by the basis module 2102 placing the electrodes at basis, default, predefined, or otherwise predetermined locations, such as locations defined by the 10-20 model.

For example, let B={b₁, . . . , bₙ} be the collection of all possible basis locations. In such an example, the only variables are the electric current running through the electrodes \vec{e}={e₁, . . . , eₙ} at these locations. In certain embodiments, a positive value indicates current flowing into the electrode, and a negative value indicates current flowing out of the electrode. Accordingly, the above formulation simplifies to:

minimize_{\vec{e}} αE_{T}(\vec{e})+βE_{D}(\vec{e}) subject to Cᵢ(\vec{e}), i=1, . . . , n

where E_{T}, E_{D}, and C, may all be functions defined on \vec{e}.

The cost module 2104 may calculate the cost of the simulation of the above formulation, the results of which may be known as a “basis simulation.” In one embodiment, the simulation solution, in current density vector, to multiple electrodes boundary condition is the weighted super-positioning of the vector solutions to all individual electrodes with unit current input. Thus, the approximation of Γ using B provides an advantage that the simulation results, with unit current, for all of the basis locations can be pre-computed and stored—the “basis simulations.” The “basis simulations,” in some embodiments, provide an appropriate starting point for the computation in each iteration of the simulation (e.g., where each iteration includes a modification of the electrode configuration by the adjustment module 2106) until an optimal threshold is reached.

In the above formulation, in one embodiment, an upper bound is placed on the number of electrodes included in the simulation. For example, the number of electrodes may be determined according to the 10-20 model. Furthermore, in certain embodiments, if, in the optimal configuration of electrodes, the current on an electrode is less than a predetermined threshold, |eᵢ|<δ, that electrode can be eliminated, which ultimately reduces the number of electrodes used in practice.

In certain embodiments, the cost module 2104 calculates the cost of the targeted stimulation for a given electrode configuration subject to various constraints on the simulated electrode configuration to determine the optimal electrode configuration. In one embodiment, a constraint on the current for an individual electrode may be determined by:

|eᵢ|<e₀ for any i=1, . . . n,

where e₀ is a safety electric current upper bound on individual electrodes.

In a further embodiment, a constraint on the ground electrode, e.g., the common ground that provides excessive or deficit current, may be determined by:

$$\vert {\sum }_{i = 1}^{n} {e}_{i} \vert < {e}_{g}$$

where e_{g} is the safety electric current upper bound on the ground electrode.

In some embodiments, a global current constraint that determines the upper bound E on the total electric current allowed for a targeted stimulation treatment may be defined as:

$${\sum }_{i = 1}^{n} \vert {e}_{i} \vert < E$$

In various embodiments, an electric current constraint may be defined for Ω_{A} such that a “hard” electrical current restriction is placed on particular biological structures, or particular target areas of biological structures. If δ is the absolute maximum electric current allowed in Ω_{A}, then:

$$\frac{1}{\vert {Ω}_{A} \vert} {\int }_{{Ω}_{A}}^{n} {‖ ∇ Φ ‖}^{2} d x < δ$$

where φ is the electric potential field, ∇φ is the gradient of the electric potential field, ∥∇φ∥² is the L₂-norm, and |Ω_{A}| denotes the size of Ω_{A}, e.g., in terms of the number of pixels or voxels that define Ω_{A}.

As mentioned above, the cost module 2104 may calculate the cost of providing an electrical current to a target area of a biological structure according to one or more predefined cost functions. The one or more predefined cost functions may incorporate one or more of the configuration variables for the electrodes described above, namely the electrical current strength, the number of electrodes, the sizes and/or shapes of the electrodes, and the location of the electrodes. In some embodiments, a direction of the electrical current is also factored into the cost functions.

Below are various examples of cost functions with a corresponding discussion of each. As background, in one embodiment, a 3D resistivity map for an individual includes well-defined orderings on the voxels. For the discussion below, a single voxel is selected as the target area. In certain embodiments, it is not important to know the specific ordering of voxels, only that a single voxel is fixed.

Let φᵢ be the electric potential field as the solution for an electrode configuration with unit current at the iₜₕ basis location, described above. In other words, φᵢ is the iₜₕ basis simulation. Given a configuration for the electrodes \vec{e}={e₁, . . . , eₙ}, the corresponding simulation solution may be formulated as:

$$Φ = {\sum }_{i = 1}^{n} {e}_{i} {Φ}_{i}$$

Note that, in some embodiments, it is not necessary to run the finite element simulation because the linear combination of basis simulations is being used.

Accordingly, Ω_{T}, Ω_{A}, and Ω_{D} are each modeled by a list of voxels in the 3D volume, following the pre-defined ordering of the voxels. Thus, the following matrices may be defined:

Tᵢ=∇Φᵢ|Ω_{T}

Aᵢ=∇Φᵢ|Ω_{A}

Dᵢ=∇Φᵢ|Ω_{D}

More specifically, for example, if Ω_{T} contains n_{T} voxels, then Tᵢ is an n_{T}×3 matrix, where each row corresponds to the current density vector at one voxel location within Ω_{T}, and the columns corresponds to the x-, y-, and z-components respectively. Accordingly, the three columns of T may be denoted as Tᵢ^{x}, Tᵢ^{y}, and Tᵢ^{z}.

In one example embodiment of a cost function, the prescribed electrical current direction and strength may be specified for the target area, such as an area of the brain. The prescribed current direction and strength may be formulated as a vector, with the length describing the current strength and the direction describing the current direction. If \vec{t} is the prescribed current density in Ω_{T}, then the cost functions may be formulated as:

$${E}_{T} ( \vec{e} ) = \frac{1}{\vert {Ω}_{T} \vert} {\int }_{{Ω}_{T}}^{n} {‖ ∇ Φ - \vec{t} ‖}^{2} d w = \frac{1}{\vert {Ω}_{T} \vert} {\sum }_{v ∈ {Ω}_{T}} {‖ {\sum }_{i = 1}^{n} {e}_{i} {T}_{i} ( v ) - \vec{t} ‖}^{2}$$

$${E}_{D} ( \vec{e} ) = \frac{1}{\vert {Ω}_{D} \vert} {\int }_{{Ω}_{D}}^{n} {‖ ∇ Φ ‖}^{2} d w = \frac{1}{\vert {Ω}_{D} \vert} {\sum }_{v ∈ {Ω}_{D}} {‖ {\sum }_{i = 1}^{n} {e}_{i} {D}_{i} ( v ) ‖}^{2}$$

In another example embodiment of a cost function, the prescribed electrical current direction may be specified for the target area. In such an embodiment, the goal may be to achieve as strong of a current strength as possible for the given direction and as allowed by the safety constraints outlined above. If \vec{u} is the unit vector prescribed for the current direction, then the cost functions may be formulated as:

$${E}_{T} ( \vec{e} ) = \frac{1}{\vert {Ω}_{T} \vert} {\int }_{{Ω}_{T}}^{n} ∇ Φ \cdot \vec{u} d w = \frac{1}{\vert {Ω}_{T} \vert} {\sum }_{v ∈ {Ω}_{T}} {\sum }_{i = 1}^{n} {e}_{i} {T}_{i} ( v ) \cdot \vec{u}$$

$${E}_{D} ( \vec{e} ) = \frac{1}{\vert {Ω}_{D} \vert} {\int }_{{Ω}_{D}}^{n} {‖ ∇ Φ ‖}^{2} d w = \frac{1}{\vert {Ω}_{D} \vert} {\sum }_{v ∈ {Ω}_{D}} {‖ {\sum }_{i = 1}^{n} {e}_{i} {D}_{i} ( v ) ‖}^{2}$$

In a further example embodiment of a cost function, the prescribed current strength may be specified for the target area. Accordingly, if t denotes the prescribed current strength, then the cost functions may be formulated as:

$${E}_{T} ( \vec{e} ) = \frac{1}{\vert {Ω}_{T} \vert} {\int }_{{Ω}_{T}}^{n} {( ‖ ∇ Φ ‖ - t )}^{2} d w = \frac{1}{\vert {Ω}_{T} \vert} {\sum }_{v ∈ {Ω}_{T}} {( ‖ {\sum }_{i = 1}^{n} {e}_{i} {T}_{i} ( v ) ‖ - t )}^{2}$$

$${E}_{D} ( \vec{e} ) = \frac{1}{\vert {Ω}_{D} \vert} {\int }_{{Ω}_{D}}^{n} {‖ ∇ Φ ‖}^{2} d w = \frac{1}{\vert {Ω}_{D} \vert} {\sum }_{v ∈ {Ω}_{D}} {‖ {\sum }_{i = 1}^{n} {e}_{i} {D}_{i} ( v ) ‖}^{2}$$

In one example embodiment of a cost function, the strongest current strength in Ω_{T} may be desired subject to the safety constraints outlined above. In such an embodiment, the cost functions may be formulated as:

$${E}_{T} ( \vec{e} ) = \frac{1}{\vert {Ω}_{T} \vert} {\int }_{{Ω}_{T}}^{n} {( ‖ ∇ Φ ‖ )}^{2} d w = - \frac{1}{\vert {Ω}_{T} \vert} {\sum }_{v ∈ {Ω}_{T}} {‖ {\sum }_{i = 1}^{n} {e}_{i} {T}_{i} ( v ) ‖}^{2}$$

$${E}_{D} ( \vec{e} ) = \frac{1}{\vert {Ω}_{D} \vert} {\int }_{{Ω}_{D}}^{n} {‖ ∇ Φ ‖}^{2} d w = \frac{1}{\vert {Ω}_{D} \vert} {\sum }_{v ∈ {Ω}_{D}} {‖ {\sum }_{i = 1}^{n} {e}_{i} {D}_{i} ( v ) ‖}^{2}$$

The foregoing formulations are examples of different cost functions that the cost module 2104 may use, based on input from a domain expert, for example, to determine a cost of a targeted stimulation given a configuration of electrodes. The cost of the targeted stimulation may be optimized by iterating through various configurations of the electrodes, e.g., varying current strength and/or direction, electrode locations, electrode size and/or shape, the number of electrodes, or the like, during a simulation and re-calculating the cost to achieve an optimal cost or to meet a threshold cost.

The adjustment module 2106, in one embodiment, is configured to modify the configuration of each electrode in response to the calculated cost not satisfying a minimum cost threshold in order to minimize the cost of transmitting the electrical current towards the target area. As described above, the modified configuration clustering electrical current transmitted by each of the electrodes at the target area according to a resistivity map of the biological structure such that the cost of transmitting the electrical current is minimized. For example, the adjustment module 2106 may modify the locations of electrodes on a person's scalp from their initial placement according to the 10-20 model to new locations based on the resistivity map for the individual. Similarly, the adjustment module 2106 may modify an electrode's size and/or shape, the current strength for the electrode, the total number of electrodes, or the like.

The adjustment module 2106 and/or the basis module 2102, in certain embodiments, places electrodes within the biological structure as well as without the biological structure. For example, the adjustment module 2106 may modify a location of one or more electrodes to be within a person's head instead of on the scalp of the person's head.

The adjustment module 2106 may compare the results from a previous simulation to determine a new configuration for the electrodes. For example, the adjustment module 2106 may check the cost, results, or the like associated with a previous configuration and, based on the results, modify the configuration of electrodes. In one embodiment, the cost module 2104 calculates an initial cost of the base configuration, and subsequent configurations, and stores the calculated initial and subsequent costs to use for comparisons in subsequent calculations. The adjustment module 2106 may use the previous results or costs as a hint, suggestion, or the like to determine a new configuration of electrodes. It should be noted that in certain embodiments, the adjustment module 2106 may increase the number of electrodes used to stimulate the target area if an increase in the number of electrodes decreases the cost of the targeted stimulation. Thus, it may not always be the case that a decrease in cost is associated with a decrease in the number of electrodes being used to stimulate the target area.

The cost module 2104, in certain embodiments, re-calculates the cost of the targeted stimulation based on the new configuration determined by the adjustment module 2106, and compares the new cost to the previously calculated cost to determine whether the new configuration is a better configuration (e.g., a less expensive configuration), an optimal configuration, or the like. In certain embodiments, the adjustment module 2106 modifies the electrode configurations and the cost module 2104 re-calculates the cost of the configuration until a threshold cost is met, achieved, reached, exceeded, or the like, a threshold number of iterations is reached, or the like. For example, the adjustment module 2106 may modify the electrode configuration on a subject's scalp for a targeted stimulation of the brain, and the cost module 2104 may re-calculate the cost of the configuration for twenty iterations and/or until the cost reaches a threshold cost.

The display module 2108, in one embodiment, is configured to graphically present the configuration of electrodes that minimizes the cost of directing the electrical current towards the target area, e.g., the determined optimal configuration for the target area. The display module 2108, in some embodiments, presents a list of electrodes and the corresponding configurations, including size and/or shape, current strength, and locations on or within the biological structure, that comprise the optimal solution. In certain embodiments, the display module 2108 presents a graphical representation of the biological structure and illustrates a configuration of the electrodes on the graphical representation.

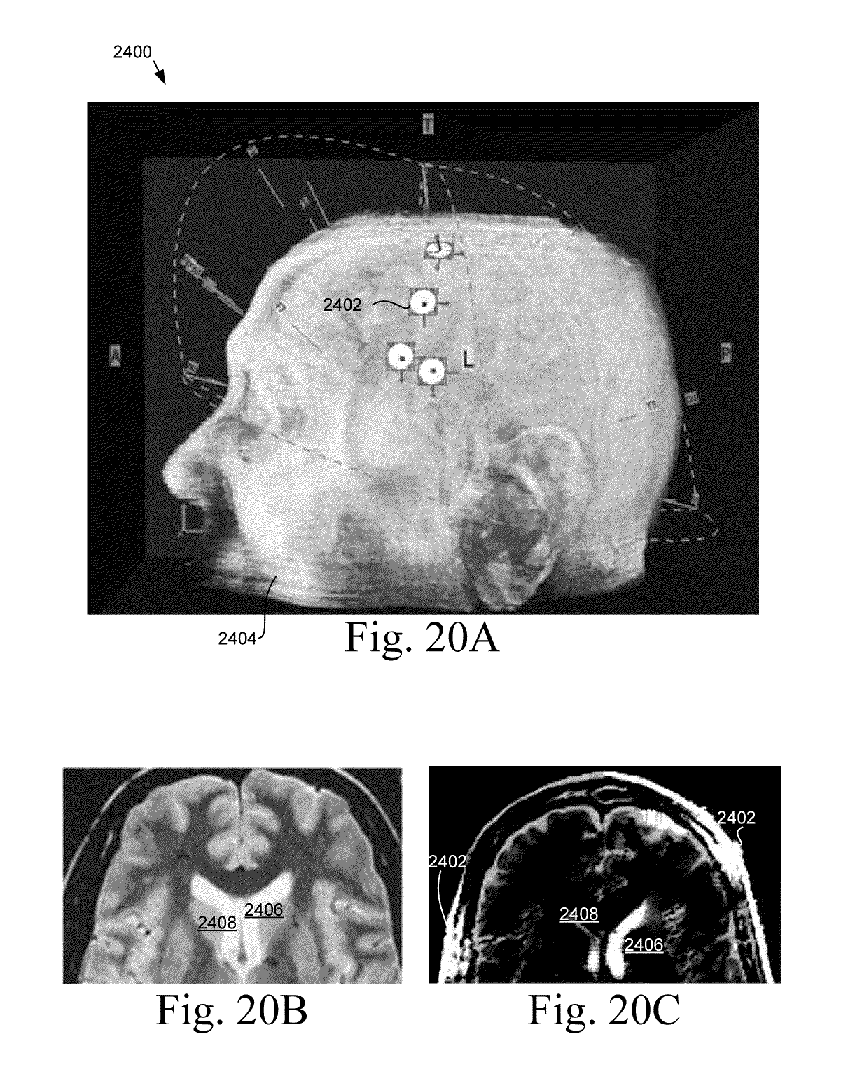

For example, as shown in the embodiment 2400 of FIG. 20A, if the target area comprises an area of the subject's brain, the display module 2108 may present an image of a subject's head 2404 that includes placement, and other characteristics, for a configuration of the electrodes 2402 that was determined to be the most cost effective, e.g., produce the lease cost or is otherwise optimal based on the objectives of the domain expert. In other words, the configuration of the electrodes depicted in FIG. 20A may be the most effective configuration for stimulating a specific area of the brain with an electrical current, by clustering electrical current at or near the target area based on the resistivity map for the individual, while avoiding other areas of the brain that are not of interest based on the domain expert's objectives, e.g., strongest current possible subject to safety constraints, current directed in a specific direction, or the like.

The display module 2108 may graphically present the results in an interface, such as a mobile application, a browser, an image viewer, or the like. The display module 2108, in a further embodiment, may provide an interactive display of the biological structure and/or the electrode configuration. For example, referring to FIG. 20A again, the display module 2108 may allow a user to interact with the display to rotate the subject's head, or the like, using an input element like a finger, stylus, mouse, or the like.

Based on the electrode configuration depicted in FIG. 20A, for example, the targeted stimulation approach described herein may allow stimulation of brain tissues (or similar tissues of the biological structure), such as the caudate 2408 and ventricles 2406 of the brain, as shown in FIG. 20B, by taking advantage of electrical clustering based on the resistivity map for the person. For example, as illustrated in FIG. 20C, the optimal placement of electrodes 2402 on or within the person's head 2404, as shown in FIG. 20A, may allow an electrical current to flow from the electrodes to be concentrated in a ventricle 2406 and then drawn into the caudate 2408 (as illustrated by the white areas in FIG. 20C. It should be noted that, in some embodiments, the white areas of FIG. 20C, e.g., the areas representing the ventricle 2406 and caudate 2408, comprise areas of high current density) by using electrical clustering with the placement of several electrodes placed on or within the person's scalp, without unnecessarily stimulating other areas of the brain that are not the target of the electrical stimulation.

In some examples, the display module 2108 allows a user to change the configuration of the electrodes in real-time by interacting with the electrodes. For example, a domain expert may change the size and/or shape of an electrode, the location of an electrode, add or remove electrodes, or the like. In response to the user's modifications, the cost module 2104 may calculate the cost of the user-defined configuration and present a comparison of the new cost to the previous cost, in real-time.

The map module 2110, in one embodiment, generates the resistivity map for the biological structure of an individual, including the target area, using one or more MRI images. In certain embodiments, the MRI images include T1 images, T2 images, proton density images, or the like. In some embodiments, the map module 2110 generates a 3D image of the biological structure, including the target area. In some embodiments, the map module 2110 generates the 3D image by combining a plurality of MRI images. The 3D image may include a plurality of voxels where each voxel is assigned a resistivity value. In some embodiments, a resistivity value is assigned to each voxel is determined according to histogram data derived from the plurality of MRI images that comprise the composite 3D resistivity map. The target area, in such an embodiment, may comprise one or more selected voxels.

The basis module 2102 and/or the adjustment module 2106, in one embodiment, may use the resistivity map to determine a configuration of electrodes to stimulate the target area at a minimal cost by determining, for example, an appropriate placement of the electrodes, a number of electrodes, a size and/or shape of each electrode, and a current strength for the electrodes based on the resistivity values of the biological materials around, surrounding, proximate to, or the like, the target area.

As discussed above with reference to FIG. 7A, certain biological materials are more resistant to electrical current, which may cause the electrical current to circumvent the more resistant material, while other biological materials are less resistant to electrical current, which may cause the electrical current to be drawn to or within the less resistant material. Accordingly, the current flowing through certain areas of medium resistivity may be manipulated, focused, clustered, or the like to be directed towards a target area. As such, by being able to determine a configuration that focuses the electrical current on the target area of a biological structure, such as a caudate of the brain, while avoiding other areas of the brain, e g , minimizing the cost of the electrical stimulation, using the resistance values derived from the resistivity map, a more focused and clustered electrical stimulation may be achieved without stimulating unintended areas of the biological structure and possibly causing negative side effects by unnecessary stimulation of the unintended areas.

FIG. 19, in one embodiment, depicts one embodiment 2300 of targeted stimulation versus standard stimulation. As shown in FIG. 19, a subject's brain is depicted under targeted electrical stimulation 2301 and standard electrical stimulation 2303. Under the standard stimulation, such as an electrode configuration that uses a 10-20 system with an input of 2 mA of current, a large portion 2304 of the subject's scalp and brain is stimulated, even though only a small target area 2305 may have been selected for electrical stimulation. On the other hand, under the targeted approach described herein, a much smaller portion 2302 of the subject's scalp and brain is stimulated due to the customized configuration of electrodes for the subject based on the resistivity map for the subject's head/brain. In certain embodiments, using the targeted approach, a smaller amount of current may be used to achieve the desired stimulation of the target area. For example, the targeted stimulation in FIG. 19 may be accomplished using only 0.3 mA of current.

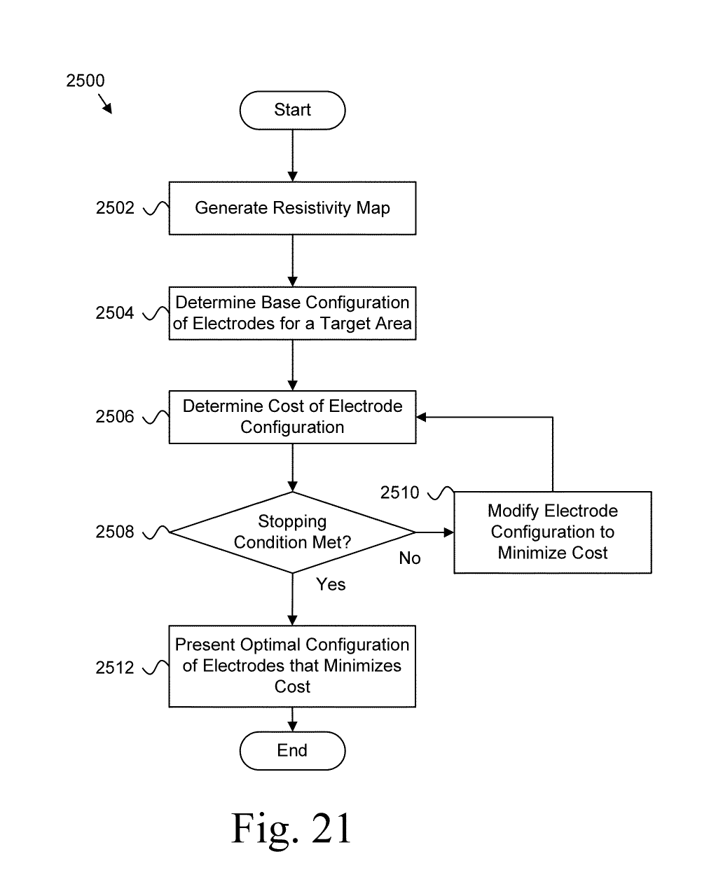

FIG. 21 illustrates one embodiment of a method 2500 for targeted electrical stimulation. In one embodiment, the method 2500 begins and a map module 2110 generates 2502 a resistivity map using one or more MRI images of a biological structure that includes the target area. The map module 2110, in certain embodiments, generates 2502 a 3D resistivity map for the biological structure where the target area is represented by one or more voxels.

In one embodiment, a basis module 2102 determines 2504 a base configuration for a plurality of electrodes configured to transmit an electrical current towards a target area of the biological structure. The base configuration for the electrodes may include an initial location, size, shape, current strength, current direction, a total current density, and a total number of electrodes. A cost module 2104, in one embodiment, determines 2506 a cost associated with transmitting an electrical current to the target area based on the configuration. The cost, as described above, is a measurement of the current density that is transmitted to the target area and a measurement of the current density that is transmitted to an unintended or non-target area.

The cost module 2104 and/or the adjustment module 2106, in various embodiments, determines 2508 whether a stopping condition has been met. In one embodiment, the stopping condition comprises determining that the calculated cost has satisfied a predetermined threshold cost. In some embodiments, the stopping condition comprises determining whether the configuration of electrodes has been modified and the cost of the configuration has been performed a predetermined number of iterations.

If the cost module 2104 and/or the adjustment module 2106 determines 2508 that the stopping condition has not been met, the adjustment module 2106 modifies 2510 the configuration of each electrode and the cost module 2104 determines 2506 a cost associated with the new configuration of electrodes until the stopping condition is met. If the stopping condition is met, the display module 2108 graphically presents 2512 the configuration for each of the electrodes that minimizes the cost of transmitting the electrical current towards the target area, and the method 2500 ends.

The present invention is not limited to the embodiments described above herein, which may be amended or modified without departing from the scope of the present invention, which is as set forth in the appended claims and structural and functional equivalents thereof. For example, the electrical property map may be used for guiding migration of stem cells. The electrical property map, and the methods of identifying the electrode sites and/or target sites can be used to generate an electric field within the biological structure to better guide the migration of stem cells.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a basis module that determines a base configuration for each of a plurality of electrodes, the electrodes configured to transmit an electrical current towards a target area of a biological structure;
a cost module that calculates a cost associated with transmitting an electrical current to the target area according to the base configuration;
an adjustment module that modifies the base configuration for each of the electrodes in response to the calculated cost not satisfying a minimum cost threshold, the modified configuration clustering electrical current transmitted by each of the electrodes at the target area according to a resistivity map of the biological structure such that the cost of transmitting the electrical current is minimized, wherein the cost module re-calculates the cost according to the modified configuration and the adjustment module modifies the configuration of each of the electrodes until the minimum cost threshold is satisfied; and
a display module that graphically presents the configuration for each of the electrodes that minimizes the calculated cost of transmitting the electrical current towards the target area.

2. The apparatus of claim 1, wherein the base configuration comprises placing each of the plurality of electrodes at predetermined locations, a predetermined location for each of the plurality of electrodes being determined according to a 10-20 model for an individual's head.

3. The apparatus of claim 2, wherein the cost module calculates an initial cost according to the base locations of the electrodes, and wherein the cost module stores the calculated initial cost for use in comparisons to subsequent cost calculations.

4. The apparatus of claim 1, wherein the adjustment module modifies a configuration of each electrode in real-time according to user input, the user input received in response to a user interacting with one or more electrodes graphically presented on a graphical representation of the biological structure.

5. The apparatus of claim 1, wherein the cost module calculates the cost according to a predefined cost function, the predefined cost function being specified in terms of one or more of a strength of an electrical current and a direction of an electrical current.

6. The apparatus of claim 5, wherein the cost module calculates the cost according to the predefined cost function subject to one or more constraints, the one or more constraints comprising one or more of an upper bound and a lower bound on:
an electrical current going through each of the plurality of electrodes;
a total electrical current going through the electrodes;
a current density in a specific area; and
a total number of electrodes being used.

7. The apparatus of claim 5, wherein the cost module calculates the cost as a function of a cost associated with the target area and a cost associated with one or more areas outside the target area, and wherein the adjustment module modifies the configuration of each of the plurality of electrodes to minimize the cost associated with the target area and the cost associated with one or more areas outside the target area.

8. The apparatus of claim 1, wherein the configuration for each of the electrodes comprises one or more of a location for each electrode, a current strength generated by the electrodes, a total number of electrodes, a size of each electrode, and a shape of each electrode.

9. The apparatus of claim 1, further comprising a map module that generates the resistivity map using one or more MRI images of an area comprising the target area, the MRI images comprising one or more of T1, T2, and proton density MRI images.

10. The apparatus of claim 9, wherein the resistivity map is a three-dimensional image comprising a plurality of voxels, the three-dimensional image being generated using a plurality of MRI images, each voxel being assigned a resistivity value according to histogram data derived from the plurality of MRI images comprising the resistivity map.

11. The apparatus of claim 10, wherein the target area comprises one or more selected voxels of the resistivity map.

12. The apparatus of claim 1, wherein the target area comprises an area within an individual's brain determined to be a candidate for electrical stimulation.

13. The apparatus of claim 1, wherein at least a portion of the basis module, the cost module, and the adjustment module comprise one or more of hardware and executable code, the executable code stored on one or more computer readable storage media.

14. A method comprising:
determining, by a processor, a base configuration for each of a plurality of electrodes, the electrodes configured to transmit an electrical current towards a target area of a biological structure;
calculating a cost associated with transmitting an electrical current to the target area according to the base configuration;
modifying the base configuration for each of the electrodes in response to the calculated cost not satisfying a minimum cost threshold, the modified configuration clustering electrical current transmitted by each of the electrodes at the target area according to a resistivity map of the biological structure such that the cost of transmitting the electrical current is minimized, wherein the cost is re-calculated according to the modified configuration and the configuration of each of the electrodes is modified until the minimum cost threshold is satisfied; and
graphically presenting the configuration for each of the electrodes that minimizes the calculated cost of transmitting the electrical current towards the target area.

15. The method of claim 14, wherein each of the plurality of electrodes are initially placed at predetermined locations, a predetermined location for each of the plurality of electrodes being determined according to a 10-20 model for an individual's head.

16. The method of claim 14, wherein a configuration of each electrode is modified in real-time according to user input, the user input received in response to a user interacting with one or more electrodes graphically presented on a graphical representation of the biological structure.

17. The method of claim 14, wherein the cost is calculated according to a predefined cost function, the predefined cost function being specified in terms of one or more of a strength of an electrical current and a direction of an electrical current.

18. The method of claim 17, wherein cost is calculated according to the predefined cost function subject to one or more constraints, the one or more constraints comprising one or more of an upper bound and a lower bound on:
an electrical current going through each of the plurality of electrodes;
a total electrical current going through the electrodes;
a current density in a specific area; and
a total number of electrodes being used.

19. The method of claim 14, wherein the cost is calculated as a function of a cost associated with the target area and a cost associated with one or more areas outside the target area, and wherein the configuration of each of the plurality of electrodes is modified to minimize the cost associated with the target area and the cost associated with one or more areas outside the target area.

20. A computer program product comprising a computer readable storage medium having program code embodied therein, the program code readable/executable by a processor for:
determining a base configuration for each of a plurality of electrodes, the electrodes configured to transmit an electrical current towards a target area of a biological structure;
calculating a cost associated with transmitting an electrical current to the target area according to the base configuration;
modifying the base configuration for each of the electrodes in response to the calculated cost not satisfying a minimum cost threshold, the modified configuration clustering electrical current transmitted by each of the electrodes at the target area according to a resistivity map of the biological structure such that the cost of transmitting the electrical current is minimized, wherein the cost is re-calculated according to the modified configuration and the configuration of each of the electrodes is modified until the minimum cost threshold is satisfied; and
graphically presenting the configuration for each of the electrodes that minimizes the calculated cost of transmitting the electrical current towards the target area.
